(19) United States
(12) Patent Application Publication
(10) Pub. No.: US 2007/0264319 A1
(43) Pub. Date: Nov. 15, 2007

(54) Transdermal Antiemesis Delivery System, Method and Composition Therefor

(76) Inventors: David B. Lebo, Warminster, PA (US); Juny Lee, Monmouth Junction, NJ (US); Je Phil Ryoo, Princeton, NJ (US); Oliver J. Toigo III, Churchville, PA (US); Francis Cupo, Yardley, PA (US)

Correspondence Address: OLSON & HIERL, LTD., 20 NORTH WACKER DRIVE, 36TH FLOOR, CHICAGO, IL, 60606, US

(21) Appl. No.: 11/661,334

(22) PCT Filed: Sep. 1, 2005
(86) PCT No.: PCT/US05/31023

§ 371 Date: Feb. 27, 2007

Related U.S. Application Data
(60) Provisional application No. 60/606,272, filed on Sep. 1, 2004.

(51) Int. Cl. A61K 31/44; A61K 47/00; A61K 47/14; A61K 47/44; A61K 9/70; A61P 1/08

(52) U.S. Cl. 424/449; 514/304; 514/772.7; 514/782; 514/785

(57) ABSTRACT

A transdermal antiemesis system, preferably in the form of a drug-in-adhesive matrix patch, is disclosed comprising an effective antiemesis amount of a selective serotonin (5-HT₃) receptor antagonist as an antiemetic incorporated in a skin adhesive composition containing at least one permeation enhancer and a water-insoluble pressure-sensitive adhesive. A preferred antiemetic is granisetron base. The transdermal antiemesis system of this invention provides controlled release of the active antiemetic ingredient from the skin-contacting adhesive formulation of a transderrnal patch, and maintains a sustained transdermal delivery of the antiemetic.

### CROSS-REFERENCE TO RELATED APPLICATION

This application claims the benefit of U.S. Provisional Application for Patent Ser. No. 60/606,272 filed on Sep. 1, 2004, incorporated herein by reference.

### TECHNICAL FIELD OF THE INVENTION

This invention relates generally to drug delivery systems, and more particularly, to the transdermal delivery of antiemetic, a transdermal antiemesis composition and method therefor.

### BACKGROUND OF THE INVENTION

Nausea and vomiting associated with cancer chemotherapy and radiotherapy is difficult to control. The therapies include the use of dopamine antagonists, such as high dose metoclopramide, and currently, selective 5-hydroxytryptamine₃ (5-HT₃) receptor antagonists. Compared to classic antiemetic medicines, such as vitamin B₆ or dexamethasone, the 5-HT₃ receptor antagonists are more potent.

Serotonin receptors of the 5-HT₃ type are located peripherally on vagal nerve terminals and centrally in the chemoreceptor trigger zone of the area postrema. During chemotherapy-induced vomiting, mucosal enterochromaffin cells release serotonin, which stimulates 5-HT₃ receptors. This evokes vagal afferent discharge, inducing vomiting. The 5-HT₃ antagonists are believed to block serotonin stimulated vomiting reflex by both peripheral and central mechanisms. The 5-HT₃ receptor antagonists suitable as antiemetic drugs include granisetron, ondansetron, tropisetron, dolasetron, and the like, and are generally administered intravenously, or orally.

Ondansetron hydrochloride, (ZOFRAN®, GlaxoSmithKline), solution is given by intramuscular or slow intravenous injection (i.v.) or orally in solution or tablet dosage form. Ondansetron base (ZOFRAN® ODT ) is given orally in tablet formulations that disintegrate rapidly on the tongue. Due to the limited half-life of ondansetron hydrochloride of about 3 to about 5 hours, it has to be administered either continuously by i.v. injection, or several times per day orally with the absolute bioavailability of 56%.

Granisetron hydrochloride, (KYTRIL®, marketed by Roche Laboratories, Inc.), can be administered by intravenous infusion or given by mouth in tablet or oral solution dosage form. Granisetron hydrochloride injection provides major benefits to millions of patients who want to prevent nausea and vomiting associated with initial and repeat cancer chemotherapy, including high dose cisplatin. The pharmacokinetic profile of granisetron hydrochloride as an oral dosage form has been widely studied. The half-life of granisetron hydrochloride is about 3 to about 4 hours in healthy adults and is about 9 to about 12 hours in cancer patients with hepatic impairment. Comparatively, granisetron hydrochloride is about 5 to about 11 times more potent than ondansetron hydrochloride.

Intravenous administration must be performed under medical supervision, however, and causes significant discomfort to the patient. Oral administration has disadvantages associated with the need for frequent administration during the day and is difficult to use as a means of drug delivery to a patient who is already suffering severe nausea and vomiting. Likewise, oral administration is not desirable for patients having difficulty in swallowing, such as very young and elderly patients, as well as patients suffering from neck, mouth or head cancer. Rectal administration, as in suppository form, avoids, to some extent, the disadvantages of oral administration, but is not convenient or a widely accepted approach to drug delivery.

Due to the inconvenience and low bioavailability of the current therapies, therefore, an effective transdermal antiemesis delivery system for a selective 5-HT₃ receptor antagonistic drug would be very convenient to use, especially for patients having chemotherapy, radiotherapy or postoperative nausea and vomiting.

Transdermal drug delivery systems in the form of patches or tapes containing medicaments have been developed and are rapidly gaining in popularity and increasing usage. A general discussion of transdermal drug delivery patch systems, measurement of skin permeation, and methods of manufacturing conventional transdermal patch devices is found in Cleary, Chapter 11, entitled “Transdermal Drug Delivery,” in Zatz Ph.D. (ed.), Skin Permeation Fundamentals and Application, pp 207-237, published by Allured Publishing Corporation, Carol Stream, Ill. (1993), the relevant disclosures of which are incorporated herein by reference.

The use of a transdermal composition, for example, a pressure-sensitive adhesive containing a drug, as a means of controlling drug delivery through the skin at essentially a constant rate, is well known. Such known delivery systems, utilizing matrix patch technology, generally incorporate the medicament into a carrier such as a polymeric matrix and/or a pressure-sensitive adhesive formulation. The pressure-sensitive adhesive must adhere effectively to the skin and permit migration of the medicament from the carrier through the skin and into the bloodstream of the patient throughout the period of therapy.

Prior attempts to transdermally delivery a therapeutically effective amount of ondansetron required a relatively high flux of the drug across the skin. In order to maintain a high flux, a high drug loading was required which could only be generally achieved by the use of so-called “reservoir patches”. Reservoir patches can advantageously contain solutions of drugs that allow drug loadings higher than those normally achieved with matrix patch technology but have many disadvantages.

Reservoir patches, because of the volume of the reservoir contents, and construction, are normally physically and visually bulky, and are cosmetically unacceptable to many patients as the patch is not flush with the skin surface when applied. Another disadvantage is that the adhesive area is limited to the periphery of the effective drug area of the patch and generally does not adhere well to the skin.

In contrast, matrix patches adhere well to the skin, and can conform to the skin contour. Also, the effective area of the patch is coterminous with the adhesive. Thus, matrix patches are more desirable than reservoir patches, but where a relatively high drug loading is required, to date, matrix patches have been undesirably large and unwieldy.

WO03/013482 A1 discloses adhesive dressings for delivery of ondansetron hydrochloride or granisetron hydrochloride which reportedly contained enhanced quantities of the drug in a cross-linked block copolymer adhesive matrix, but the adhesive requires that a high molecular weight, oily ester plasticizer be present in an amount of at least 10% by weight of the cross-linked block copolymer. Additionally, the salts of the drugs, especially ondansetron, reportedly were delivered significantly better than the free base form of the compounds.

WO98/53815 discloses a transdermal drug delivery device for tropisetron or granisetron which requires that the adhesive layer have an acrylate or methacrylate co-polymer that includes a hydrophilic co-monomer, free of nucleophilic groups, necessary for solubilizing the drug.

WO2004/069141 A2 discloses adhesive patches for the transdermal administration of granisetron at levels above 4%, or ramosetron, which require that the adhesive have non-acidic hydroxyl groups and teaches away from using penetration enhancers to avoid increasing transdermal flux. The patches reportedly have a lag time of about 2 hours, dispense the granisetron in a substantially uneven form, and flux was reportedly sufficiently high to deplete the device of granisetron after only 24 hours, thus requiring more than one patch daily for providing a continuous high level of drug.

There is an ongoing need and desire, therefore, for a convenient antiemesis drug delivery device that can provide controlled and sustained transdermal release of an antiemetic 5-HT₃ receptor type antagonistic drug, and of granisetron in particular, at relatively lower drug concentrations and at higher permeation rates than those provided by prior art devices. The present invention provides such a transdermal drug system.

### SUMMARY OF THE INVENTION

The present invention provides a transdermal antiemesis system, a transdermal, skin adhesive antiemesis composition, and a method for transdermal delivery of an effective antiemesis amount of a selective serotonin (5-HT₃) receptor antagonist. A preferred transdermal antiemesis system is a single-layer, drug-in-adhesive matrix patch system that provides controlled release of an effective amount of an antiemetic 5-HT₃ receptor antagonist drug from a skin-contacting adhesive formulation of the patch, and maintains a sustained transdermal delivery of the antiemetic drug during the therapy regimen. A preferred antiemetic 5-HT₃ receptor antagonist is granisetron.

A preferred transdermal skin adhesive antiemesis composition for use as a drug-in-adhesive matrix comprises, on a total composition weight basis, a therapeutically effective antiemesis amount of granisetron base; a physiologically skin tolerable, substantially water-insoluble, pressure sensitive adhesive; and an amount of at least one penetration enhancer effective to provide a substantially uniform, sustained release of drug from the composition. Preferably, granisetron base has a solubility in the range of at least about 10 to at least 200 micrograms per milliliter (μg/ml) in the penetration enhancer at an ambient room temperature in the range of about 20 to about 25° C.

A preferred transdermal antiemesis patch comprises a substantially inert, drug-impermeable backing film and a therapeutically effective antiemesis amount of skin adhesive antiemesis composition of the invention coated thereon to provide a single layer, drug-in-adhesive skin contacting adhesive surface. Preferably, the transdermal patch includes a protective release liner laminated to the skin-contacting adhesive surface and through which the antiemetic drug is not permeable. For use, the release liner is removed and the skin contacting adhesive surface of the transdermal patch is applied to the skin, preferably in a substantially hairless area such as the skin of the chest, back, waist, or upper arm.

A presently preferred occlusive backing preferably has a moisture vapor transmission rate (MVTR) of not more than about 200 g/m²/24 hours, more preferably of not more than about 75 g/m²/24 hours and most preferably of not more than about 20 g/m²/24 hours. A preferred transdermal patch has a practical active surface area sized in the range of about 10 to about 20 cm² and delivers antiemetic drug at a rate in the range of least about 4 to about 15 micrograms/square centimeter per hour of therapy.

A preferred transdermal skin adhesive antiemesis composition for use in a matrix transdermal delivery patch of the invention comprises, on a total drug-in-adhesive matrix composition weight basis;

granisetron base in the range of about 1 to about 10 weight percent, more preferably in the range of about 2 to about 6 weight percent, most preferably in the range of about 3 to about 5 weight percent;

at least one skin penetration enhancer in a total amount in the range of about 0.1 to about 95 weight percent, more preferably in the range of about 1 to about 50 weight percent, most preferably in the range of about 2 to about 20 weight percent;

the remainder comprising an effective skin adhesive amount of a substantially water-insoluble, polymeric pressure sensitive adhesive.

Preferred adhesives can be acrylic acid based, rubber based, or silicone based. Most preferred is a water-insoluble, acrylic acid based adhesive.

The transdermal antiemesis compositions and delivery system provide controlled release of the active antiemetic ingredient and can be produced by a variety of methods known in the preparation of drug-in-adhesive matrices, including the mixing of drug, at least one permeation enhancer, an adhesive, solvent processing aids, and additional optional excipient ingredients in solution, followed by removal of the processing solvents.

Advantageously, transdermal antiemesis patches of this invention may be tailored to deliver antiemetic drug in an effective antiemesis amount in the range of about 1 to about 2 milligrams per day to preferably provide a once-a-day or twice-a-week or weekly therapeutic treatment regimen. The transdermal antiemesis system preferably ameliorates nausea and vomiting associated with radiation, including total body irradiation and fractionated abdominal radiation and chemotherapy-induced emesis.

### BRIEF DESCRIPTION OF THE DRAWINGS

In the drawings,

FIG. 1 is a schematic illustration of a transdermal antiemesis patch of the present invention represented as a single-layer drug-in-adhesive delivery system;

FIG. 2 is a graphical representation of release of granisetron from transdermal skin adhesive antiemesis systems of Examples 5-B, 5-C, 5-D and 5-E over a period of about 24 hours;

FIG. 3 is a graphical representation of in vitro skin permeation (human cadaver) in micrograms per square centimeter of granisetron from transdermal skin adhesive antiemesis systems of Examples 5-K and 8-J over a period of about 24 hours;

FIG. 4 is a graphical representation of in vitro skin permeation (human cadaver) of granisetron in average micrograms per square centimeter from transdermal skin adhesive antiemesis systems of Examples 15-J, 17-B, 17-C, 17-D, and 17-E; and

FIG. 5 is a graphical representation of drug release profiles over a period of about 24 hours from transdermal antiemesis systems of Examples 15-J, 17-A, 17-B, 17-C, and 17-D in average micrograms per square centimeter plotted as a Square-Root-of-Time (hrs.).

### DETAILED DESCRIPTION OF PREFERRED EMBODIMENTS

Selective serotonin (5-HT₃) receptor antagonists known in the art to be useful as antiemetic drugs include: granisetron, tropisetron, ondansetron, dolasetron, azasetron, polonosetron, ramosetron, and the like. The transdermal antiemesis delivery system is described herein using granisetron as a preferred antiemetic, but is not intended to be limited thereto.

Granisetron is a particularly preferred selective serotonin (5-HT₃) receptor antagonist used for the treatment of nausea and vomiting, and to prevent chemotherapy-induced emesis. Granisetron is designated chemically as 1-methyl-N-[(3-endo)-9-methyl-9-azabicyclo[3.3.1]non-3-yl]-1H-imidazole-3-carboxamide, has a molecular weight of 312.4, and the molecular formula: C₁₈H₂₄N₄O. The preparation of granisetron is described in U.S. Pat. No. 4,886,808, incorporated herein by reference. Granisetron is therapeutically active in the free base form, as well as in pharmaceutically acceptable acid addition salts thereof. The free base form of granisetron is generally more active than the salt form, but is less soluble in conventional therapeutical vehicles. Granisetron hydrochloride, for example, is a white to off-white solid, having a molecular weight of 348.9, a reported melting point in the range of about 290 to about 292° C. and is soluble in water and normal saline at 20° C.

The term “granisetron,” as used herein, includes the free base form of this compound, as well as pharmaceutically acceptable acid addition salts thereof. Granisetron base is particularly preferred for the antiemesis embodiments of the present invention.

Conventionally, granisetron is given in a typical oral dose of about 1 to about 2 mg twice daily, to provide an oral bioavailability of granisetron in the range of about 50 to about 60%, and an elimination half-life of about 7 hours.

It was found that a transdermal antiemesis patch of the present invention, based on a typical drug load in the range of about 3% by weight to about 5% by weight, can provide a uniform, sustained total delivery of about 8 mg granisetron drug over a period of about 4 days at an efficiency of about 80%.

The term “transdermal patch” as used herein includes structures in the form of tapes, strips, sheets, dressings or any other like form known to those skilled in the art, without limitation as to dimensional size or construction, except where specifically indicated, that can be applied to the skin of a mammal. A preferred transdermal antiemesis system embodiment, without being limited thereto, has a single-layer, drug-in-adhesive (DIA) patch anatomy, characterized by the inclusion of the drug directly within the skin-contacting adhesive medium rather than as a separate layer. In this type of transdermal patch embodiment, the adhesive medium serves to both affix the antiemesis system device to the skin and as the formulation foundation containing the drug and all excipients, and is applied as a substantially coterminous coating on a backing support film or layer. The terms “skin adhesive composition,” “drug-in-adhesive matrix,” or “DIA matrix” are used interchangeably herein to refer to the drug-containing adhesive medium of the transdermal patch. A polymeric pressure sensitive skin adhesive medium is preferred.

Transdermal granisetron delivery system embodiments of this invention provide an effective antiemesis amount of granisetron incorporated in a physiologically tolerable water-insoluble, pressure sensitive adhesive formulation and maintained in substantially dissolved (i.e., non-crystalline) form therein. The term “effective antiemesis amount” means that the concentration of the drug in the skin adhesive is such that it results in a therapeutic antiemesis level of drug delivered over the term that the dosage form is to be used; e.g., that a therapeutically effective blood level of the drug is achieved or maintained. The amount of granisetron incorporated into the adhesive medium of the skin adhesive composition can vary with the desired therapeutic dosage effect and the time period over which the transdermal drug delivery system is to provide therapy at the flux rate of the drug from the system.

Transdermal patches prepared with a transdermal granisetron skin adhesive composition of this invention can be prepared to provide any desired active dimensional surface area. A useful area may be in the range of about 1 to about 30 square centimeters (cm²), preferably in the range of about 2 to about 25 cm², more preferably in the range of about 10 to about 20 cm². Preferably the transdermal patches have a drug load in the range of about 0.01 to about 10 mg/cm², more preferably in the range of about 0.1 to about 5 mg/cm², most preferably in the range of about 0.2 to about 2 mg/cm².

A preferred transdermal drug delivery patch preferably has an active skin contacting area in the range of about 10 to about 20 square centimeters (cm²), a drug load in the range of about 5 to about 25 mg/patch, preferably in the range of about 10 to about 20 mg/patch, and delivers a drug flux in the range of at least about 4 to at least about 15 micrograms/cm²/hour (μg/cm²/hr), more preferably in the range of at least about 6 to at least about 12 μg/cm²/hr, for a period of at least one day, preferably for a period of at least three days to about a week. The actual amount of drug needed can be experimentally determined and the size of the active skin contacting area and structure of the transdermal patch can be tailored accordingly by the appropriate selection of drug penetration enhancer, and backing material.

Agents that accelerate the delivery of the drug through the skin are variously referred to in the art as skin-penetration enhancers, permeation enhancers, accelerants, adjuvants, and sorption promoters, and are collectively referred to herein as “penetration enhancers.” A preferred skin adhesive composition of the drug delivery system contains at least one penetration enhancer, and more preferably at least two penetration enhancers, where at least one penetration enhancer can dissolve granisetron base in an amount of at least about 10 μg/ml, more preferably of at least about 20 μg/ml.

A general discussion of transdermal patch drug delivery systems, measurement of skin permeation, and methods of manufacturing conventional transdermal patch devices is found in Cleary, Chapter 11, entitled “Transdermal Drug Delivery,” and general discussion of various skin penetration enhancers is in Rieger, Chapter 2, entitled “Factors Affecting Sorption of Topically Applied Substances,” both found in Zatz Ph.D. (ed.), Skin Permeation Fundamentals and Application, pp. 33-72, published by Allured Publishing Corporation, Carol Stream, Ill. (1993), the relevant disclosures of which are incorporated herein by reference. A detailed description of transdermal drug delivery systems, skin penetration enhancers and adhesives is also given in U.S. Pat. No. 6,235,306 B1, the disclosures of which are incorporated herein by reference.

Non-drug ingredients employed in the compositions discussed herein are referred to by their commonly used chemical or trade names or by the international nomenclature commonly referred to as INCI name given them in any edition of the International Cosmetic Ingredient Dictionary and Handbook, (INCI Dictionary), such as found in Volumes 1-3, of the Seventh Edition (1977) or English Edition (2000) or Ninth Edition (2002), all published by the Cosmetic, Toiletry, and Fragrance Association, Washington, D.C.

The penetration enhancer is present in an amount sufficient to enhance the skin permeation of the granisetron. The specific amount of penetration enhancer varies necessarily according to the desired release rate. The amount of total permeation enhancer in the skin adhesive composition, on a total skin adhesive composition weight basis, may be in the range of about 0.1 to about 95% by weight, more preferably in the range of about 1 to about 50% by weight, most preferably in the range of about 2 to about 20% by weight. Particularly preferred are combinations of at least two permeation enhancers. When combinations are used, each permeation enhancer may be in the range of about 1 to about 20% by weight, more preferably in the range of about 2 to about 10% by weight, based on the total weight of the skin adhesive composition. The actual amount and type of penetration enhancer is not limited as long as the penetration enhancer is compatible with the DIA matrix and does not negatively affect the physical integrity of the transdermal patch.

In general, suitable penetration enhancers can be chosen from alcohols, carboxylic acids, carboxylic esters, glycols, polyols, amides, surfactants, terpenes, alkanones, solvents, and mixtures thereof. See generally Chattaraj, et al., “Penetration Enhancer Classification”, pp. 5-20 in Maibach, et al. (eds.), Percutaneous Penetration Enhancers, CRC Press, Inc., Boca Raton, Fla. (1995), Büyüktimkin, N., et al., “Chemical Means of Transdermal Drug Permeation Enhancement,” in Ghosh, T. K., et al., (eds.) Transdermal and Topical Drug Delivery Systems, Interpharm Press, Inc., Buffalo Grove, Ill. (1997), the relevant disclosures of which are incorporated herein by reference.

Non-limiting examples of suitable alcohols include methanol, ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, oleyl alcohol, linolyl alcohol, linolenyl alcohol and mixtures thereof.

Non-limiting examples of suitable carboxylic acids include fatty acids, such as caproic, capric, caprylic, lauric, myristic, palmitic, stearic, isostearic acid, oleic, linoleic, linolenic, and the like; and other straight-chain or branched organic acids, such as valeric, heptanoic, pelargonic, isovaleric, neopentanoic, neoheptanoic, neononanoic, trimethyl hexanoic, neodecanoic and mixtures thereof.

Non-limiting examples of suitable carboxylic esters include sorbitan derivatives, such as sorbitan monolaurate (SPAN® 20, CRILL™ 1 NF), sorbitan monooleate (SPAN® 80, CRILL™ 4NF), and the like; esters of C₆-C₂₂ carboxylic acid, such as isopropyl myristate, isopropyl palmitate, octyldodecyl myristate, ethyl oleate, ethyl laurate, isopropyl n-hexanoate, isopropyl n-decanoate, isopropyl n-butyrate, methylvalerate, methylpropionate, diethyl sebacate, and the like; and acetates, such as ethyl acetate, butyl acetate, methyl acetate, and the like, and mixtures thereof. Preferred are sorbitan monolaurate and sorbitan monooleate.

Non-limiting examples of suitable glycols and polyols include propylene glycol, polyethylene glycol (PEG), ethylene glycol, diethylene glycol, triethylene glycol (TEG), dipropylene glycol, glycerol, propanediol, sorbitol, isosorbitol, dextrans, butanediol, pentanediol, hexanetriol, and mixtures thereof. Preferred are propylene glycol, and PEG, particularly a PEG having an average of about 8 ethylene oxide units (PEG-400).

Non-limiting examples of suitable surfactants include anionic surfactants, cationic surfactants, nonionic surfactants, amphoteric surfactants, bile salts and lecithin. Suitable anionic surfactants include sodium laurate, sodium lauryl sulfate, and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxypoly(oxyethylene)poly(oxypropyl) poly(oxyethylene) block copolymers, polyoxyethylene fatty ethers, polyoxyethylene sorbitan esters, polyethylene glycol esters of fatty alcohols, and mixtures thereof. Suitable α-hydro-ω-hydroxy-poly(oxyethylene) poly(oxypropyl) poly(oxyethylene) block copolymers include Poloxamers 182, 184, 231, and mixtures thereof. Suitable polyoxyethylene fatty ethers include polyoxyethylene (4) lauryl ether (BRIJ® 30), polyoxyethylene (2) oleyl ether (BRIJ® 93), polyoxyethylene (10) oleyl ether (BRIJ® 96), polyoxyethylene (20) oleyl ether (BRIJ® 99), and mixtures thereof. Suitable polyoxyethylene sorbitan esters include the monolaurate (TWEEN® 20) the monopalmitate (TWEEN® 40), the monostearate (TWEEN® 60), the monooleate (TWEEN® 80), and mixtures thereof. Suitable polyethylene glycol esters of fatty acids include polyoxyethylene (8) monostearate (MYRJ® 45), polyoxyethylene (30) monostearate (MYRJ® 51), the polyoxyethylene (40) monostearate (MYRJ® 52), and mixtures thereof. Preferred are nonionicsurfactants, particularly polyoxyethylene fatty ethers.

Suitable amphoteric surfactants include, without limitation thereto, lauramidopropyl betaine, cocamidopropyl betaine, lauryl betaine, cocobetaine, cocamidopropylhydroxysultaine, aminopropyl laurylglutamide, sodium cocoamphoacetate, sodium lauroamphoacetate, disodium lauroamphodiacetate, disodium cocoamphodiacetate, sodium cocoamphopropionate, disodium lauroamphodipropionate, disodium cocoamphodipropionate, sodium lauriminodipropionate, disodium cocoamphocarboxymethylhydroxypropylsulfate, and the like.

A preferred penetration enhancer comprises an N,N-di(C₁-C₈)alkylamino substituted, (C₄-C₁₈) alkyl (C₂-C₁₈) carboxylic ester or a pharmaceutically acceptable acid addition salt thereof. As used herein, the term “(C₄-C₁₈) alkyl (C₂-C₁₈) carboxylic ester” means an ester of a (C₄-C₁₈) alcohol and a (C₂-C₁₈) carboxylic acid. The term “N,N-di(C₁-C₈)alkylamino substituted,” in reference to a (C₄-C₁₈) alkyl (C₂-C₁₈) carboxylic ester means that either the alcohol portion or the carboxylic acid portion from which the ester is prepared bears an amino substituent NRₓR_{y}, wherein Rₓ and R_{y} are each independently a (C₁-C₈) alkyl group. Preferably Rₓ and R_{y} are both methyl groups.

Preferred are dodecyl-2-(N,N-dimethylamino)-propionate (DDAIP); dodecyl-2-(N,N-dimethylamino)-acetate (DDAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof. Particularly preferred is DDAIP, alone or in combination with an auxiliary permeation enhancer. DDAIP is available from Steroids, Ltd. (Chicago, Ill.). The preparation of DDAIP and crystalline acid addition salts thereof is described in U.S. Pat. No. 6,118,020 to Büyüktimkin, et al., which is incorporated herein by reference. Long chain similar amino substituted, alkyl carboxylic esters can be synthesized from readily available compounds as described in U.S. Pat. No. 4,980,378 to Wong, et al., which is incorporated herein by reference to the extent that it is not inconsistent herewith.

Non-limiting examples of solvents include aliphatic esters, such as triethylcitrate (TEC), and triacetin; aromatic esters, such as diethylphthalate (DEP); dipolar aprotic solvents, such as N-methyl-2-pyrrolidone (NMP), diethylene glycol monoethyl ether (DGME, transcutol), isosorbide dimethylether (DMI), dimethyldecylphosphoxide, methyloctylsulfoxide, dimethyllaurylamide, dodecylpyrrolidone, dimethylacetamide, dimethylsulfoxide, decylmethylsulfoxide, and dimethylformamide; oils, such as mineral oil, silicone oils, squalane, and octanol, and the like, which affect keratin permeability.

Surprisingly, polymers of 1-vinyl-2-pyrrolidione, such as homopolymers (povidone, polyvinylpyrrolidone (PVP)), and vinyl acetate copolymers thereof, were found useful as a skin penetration enhancer. PVP is preferred. PVP is sold in various solubility grades under the trade name, PLASDONE™ by ISP Technology.

Particularly preferred skin permeation enhancers are dipolar aprotic solvents, particularly NMP, DGME, and DMI; aliphatic esters, particularly TEC, and triacetin; carboxylic esters that are sorbitan derivatives, particularly sorbitan monolaurate (SPAN® 20, CRILL™ 1 NF), and sorbitan monooleate, N,N-di(C₁-C₈)alkylamino substituted C₄-C₁₈) alkyl (C₂-C₁₈) carboxylic esters, particularly DDAIP, nonionic surfactant, particularly PEG-4 lauryl ether (laureth-4, BRIJ® 30), oil, particularly mineral oil, PVP, particularly PVP having a K value in the range of about 25 to about 35, and combinations thereof.

Pressure-sensitive adhesives (PSAs) are well known materials that preferably can be adhered to a substrate by application of a relatively light force and, when removed, leave substantially no residue. The major classes of PSAs are composed of polymers, such as acrylics, silicones, and isobutylenes. In the present transdermal delivery system, the PSA component provides for intimate contact of the drug-containing skin adhesive and the skin surface for maintaining controlled and sustained transdermal release of the drug.

The pressure sensitive adhesive component is not limiting as long as it is physiologically tolerable to human skin and can maintain granisetron base incorporated therein in a substantially crystal-free form, i.e., no crystals are visually or microscopically observable in the adhesive formulation. In transdermal patch embodiments, the pressure sensitive adhesive may constitute an amount in the range of about 30% to about 99% of the total weight of the skin adhesive composition, preferably about 40% to about 97%, and more preferably about 50% to about 95%; the amount of adhesive being dependent on the amount of drug and other formulation components.

A preferred pressure sensitive adhesive is an acrylic-based polymer, such as any of the homopolymers, copolymers, terpolymers, and the like, of various acrylate polymers known in the pressure-sensitive adhesive arts. Exemplary acrylate polymers include, but are not limited to, polymers of one or more monomers of acrylic acids and other copolymerizable monomers. The acrylate polymers also include copolymers of alkyl acrylates and/or methacrylates and/or copolymerizable secondary monomers or monomers with functional groups. By varying the amount of each type of monomer added, the cohesive properties of the resulting acrylate polymer can be changed as is known in the art.

Acrylate monomers which are generally used include acrylic acid, methacrylic acid, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, and tridecyl methacrylate. Functional monomers, copolymerizable with the above alkyl acrylates or methacrylates, which are generally used include vinyl acetate, acrylic acid, methacrylic acid, maleic acid, maleic anhydride, hydroxyethyl acrylate, hydroxypropyl acrylate, acrylamide, dimethylacrylamide, acrylonitrile, diacetone acrylamide, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, and the like, and mixtures thereof. Detailed examples of acrylic adhesives suitable in the practice of the invention are described in Satas (ed.), “Acrylic Adhesives,” Handbook of Pressure-Sensitive Adhesive Technology, 2nd ed., pp. 396-456, Van Nostrand Reinhold, N.Y. (1989).

Other suitable adhesive materials for the skin adhesive layer may include, polysiloxanes, polyisobutylenes, polyurethanes, plasticized ethylene vinyl acetate copolymers, low molecular weight polyether amide block polymers (e.g., PEBAX), tacky rubbers, such as polyisobutene, polystyrene-isoprene copolymers, polystyrene-butadiene copolymers, and mixtures thereof.

Preferred pressure sensitive adhesive materials for use in the drug-in-adhesive matrix are polyacrylates, polystyrene-isoprene block copolymers, polyisobutylenes, silicones, and polyurethane, with acrylic-vinyl acetates, polyacrylates and tacky rubbers particularly preferred. Exemplary acrylic adhesives are commercially available and include, without limitation thereto, a series of polyacrylate adhesives sold under the trademarks GELVA® Multipolymer Solution as GMS grades 737, 788, 2873, 3067, 3071, 3083, 3235, 9073, and 9083 by UCB Group, and pressure sensitive adhesives sold under the trademarks DURO-TAK® as organic solutions series 87-2100, 87-2194, 87-2196, 87-2353, 87-2516, 87-2979, 87-9301, and the like, sold by National Starch and Chemical Corporation. An exemplary rubber adhesive is styrene-isoprene-styrene (SIS) block copolymer, available under the trademarks QUINTAC® 3433 from Nippon Zeon (Japan) and CARIFLEX™ TR-1107 from Shell Kogaku K. K.

Optionally, the skin adhesive composition may include a plasticizer or tackifying agent in the formulation to improve or modify the adhesive characteristics of the composition. Suitable tackifying agents are known in the art and generally include aliphatic hydrocarbons; mixed aliphatic and aromatic hydrocarbons; aromatic hydrocarbons; substituted aromatic hydrocarbons; hydrogenated esters; polyterpenes; rosin esters, hydrogenated wood rosins, and the like. The tackifying agent employed is preferably compatible with the adhesive. An exemplary tackifying agent is silicone fluid (e.g., 360 Medical Fluid, available from Dow Coming Corporation, Midland, Mich.), mineral oil, or rosin ester (e.g., KE-311 available from Arakawa Chemical Co., Osaka, Japan).

The transdermal antiemesis skin adhesive compositions of this invention can be produced by a variety of methods known in the preparation of drug-in-adhesive matrices, including the mixing of a polymeric adhesive, drug, volatile processing solvent, additional co-solvent, if any, permeation enhancer, and excipient ingredients into a “wet-base” solution, followed by removal of the volatile processing solvents. The drug-in-adhesive transdermal delivery system of this invention permits incorporation of therapeutically effective amounts of the antiemesis drug directly within the skin-contacting adhesive formulation of a transdermal patch, while maintaining acceptable transdermal delivery of the antiemetic, and acceptable tack, and peel adhesive properties of the delivery device.

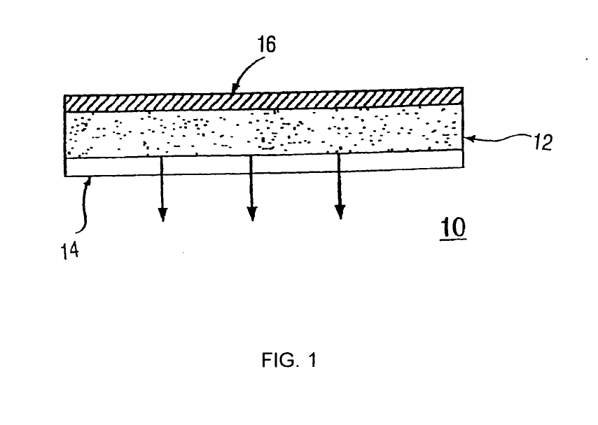

In a transdermal patch device aspect of the invention, the skin adhesive composition can comprise a single layer, drug-in-adhesive monolithic device as illustrated in FIG. 1. FIG. 1 is a schematic illustration of a single-layer drug-in-adhesive patch embodiment of the invention. The delivery system comprises a monolithic body 10 of a defined geometric shape with a protective release liner 14 on one side of the monolithic body 10 and a backing layer 16 on the other side. Removal of the release liner 14 exposes the pressure-sensitive drug-in-adhesive matrix 12 which functions both as the drug carrier matrix and as the means of applying the drug delivery system to the skin of the patient. The backing 16 is substantially coterminous with the drug-in-adhesive matrix 12. The liner 14 is substantially coterminous with the drug-in-adhesive matrix 12 and may be configured or adapted to include a finger hold for grasping and aiding in removal of the liner therefrom.

The backing functions as the primary structural element of the device and preferably provides the patch with flexibility, drape and, controlled occlusivity. The material used for the backing layer should be inert and impermeable to the drug, permeation enhancer or other desirable components of the skin adhesive matrix. The backing may be made of one or more laminate sheets or films of a flexible material that serves as a protective covering to prevent loss of drug or vehicle or both by transmission through the upper surface of the device. Preferably, the backing is sufficiently flexible to conform to the skin area on which the patch is applied and can be worn comfortably with little or no likelihood of the patch disengaging from the skin due to differences in the flexibility or resiliency of the skin and the device.

The backing material should be substantially inert, i.e., non-reactive with the ingredients of the formulations. The backing can be occlusive, semi-occlusive, or non-occlusive (breathable), depending on the amount of moisture vapor transmission rate (MVTR) desired. Methods of measuring MVTR are well known in the art, and a description is found, for example, in U.S. Pat. No. 5,702,720 to Effing, et al., the relevant disclosures of which are incorporated herein by reference. A standard value of cutaneous insensible perspiration, representing loss of water from living skin, has been regarded to be about 16 g/m²/hr. as reported in Kuno, Yas, Human Perspiration, Ch. 1, pp. 26-27, Charles C. Thomas Publisher, (Springfield, Ill., 1956). Based on this water loss value, Effing, et al., in U.S. Pat. No. 5,702,720 estimated the daily MVTR of living skin to be about 400 g/m²/24 hours (hrs). Devices that have a MVTR substantially below this value, therefore, are generally described as occlusive and those having a MVTR value substantially greater than 400 g/m²/24 hrs. are described as non-occlusive. A preferred occlusive backing has a MVTR value of not more than about 200 g/m²/24 hours; more preferably of not more than about 75 g/m²/24 hrs., and most preferably of not more than about 20 g/m²/24 hrs. A preferred substantially semi-occlusive backing has a MVTR value in the range of about 400 g/m²/24 hrs. to not more than about 1,000 g/m²/24 hrs. A preferred substantially non-occlusive backing has a MVTR value in the range of about 1,000 g/m²/24 hrs. to not more than about 1,500 g/m²/24 hrs.

Backing materials are well known in the art and can comprise films or sheets of polyesters; such as polyethylene terephthalate (PET); polyolefins, such as polyethylene, polypropylene. and copolymers thereof; vinyl acetate resins, such as ethylene/vinyl acetate copolymers (EVA), ethylene-ethylacrylate copolymer (EEA), vinyl acetate-vinyl chloride copolymers; polyamides, polyvinyl chlorides, polyvinylidene chlorides, polyurethanes, such as spandex (SPDX); celluloses, such as cellulose acetate, ethyl cellulose, cotton, and the like; metal foils, such as aluminum; and laminate combinations thereof. Backing materials can be woven fabric, non-woven fabric, elastomeric fabric, knitted fabric, spun-bonded fabric, and combinations thereof.

A useful backing material generally may have a caliper thickness in the range of about 2 micrometers to about 100 micrometers, preferably in the range of about 15 micrometers to about 70 micrometers, more preferably in the range of about 30 micrometers to about 60 micrometers.

A preferred occlusive backing material is a polyester/EVA laminate (3M™ SCOTCHPAK™ 9732). A preferred substantially semi-occlusive, breathable backing is a PET-lined non-woven fabric, and preferred substantially non-occlusive backing materials include polyurethane and non-woven fabrics.

The release liner is a disposable element which protects the device prior to application. Typically, the release liner is formed from a material impermeable to the drug, vehicle and adhesive, and is easily stripped from the skin contacting adhesive matrix. The release liner is occlusive and functions to protect the surface of the drug-in-adhesive layer during production, storage, and transport.

Suitable release liners are well known in the art and are commercially available. Release liner materials are generally occlusive sheet materials, such as webs or films of polyester, poly(vinyl chloride), poly(vinylidene chloride), polyethylene, polyethylene terephthalate (PET), ethylene/vinyl acetate copolymers (EVA) polystyrene and the like, paper (e.g., wood-free paper and glassine paper), laminate films of paper and polyolefin, metal foils and the like. Release liners are preferably subjected to a release treatment, such as silicone or fluoropolymer treatment, on the surface of the liner that comes in direct contact with the skin adhesive DIA matrix of the transdermal patch. Silicone-coated polyester, fluoropolymer-coated polyester, and silicone-coated aluminum are typically preferred. Where a polysiloxane is part of the adhesive system, the release liner must be compatible with the silicone adhesive.

Exemplary release liners include silicone coated polyester (PET) film sold by Mylan Technologies, Inc. under the trade name MEDIRELEASE®, liner films sold in various grades by Loparex Inc, and liner films sold under trade names BIO-RELEASE® and SYL-OFF® 7610 by Dow Coming Corporation. A preferred release liner is a silicone coated PET, such as the MEDIRELEASE® 2200 series available in a variety of thicknesses from Mylan Technologies, Inc., and, in particular, MEDIRELEASE® 2249 reportedly having a nominal caliper thickness of 3 mils (76.2 μm) and a liner release force value of 14 g/in. (5.6 g/cm). Another preferred release liner is Liner Grade 24950 available from Loparex Inc., reportedly having a nominal caliper thickness of 2 mils (50.8 μm) and a nominal liner release force value of 25 g/in (10 g/cm).

Other exemplary release liners include, without limitation, fluoropolymer coated polyester film sold by 3M under the trade names SCOTCHPAK™ 1022 Release Liner reportedly having a nominal caliper thickness of 3 mils (76.2 μm) and a liner release force of <100 g/in (<1.0 Newtons/25.4 millimeters (mm)) and SCOTCHPAK™ 9742 reportedly having a nominal caliper thickness of 4.6 mils (117 μm) and a liner release force of <100 g/in (<1.0 Newtons/25.4 millimeters (mm)); and SCOTCHPAK™ 9744 reportedly having a nominal caliper thickness of 3 mils (76.2 μm) and a liner release force of <100 g/in (<1.0 Newtons/25.4 millimeters (mm)).

A useful release liner generally has a thickness in the range of about 12 to about 500 micrometers, preferably in the range of about 25 to about 400 micrometers, more preferably in the range of about 50 to about 150 micrometers.

The configuration of the transdermal delivery system of the present invention can be in any shape or size as is necessary, desirable, or practical. Illustratively, a single dosage unit may have an active dosage surface area in the range of about 1 to about 30 cm². Preferred active dosage surface areas are in the range of about 2 to about 25 cm², more preferably in the range of about 10 to about 20 cm².

A transdermal patch, or individual dosage unit, of the present invention can be produced in any manner known to those of skill in the art. The skin adhesive composition is first formulated as a water-insoluble, liquid solution, referred to herein as a “wet-base” solution. After the wet-base solution is formulated, it may be cast on a support, such as a release liner, substantially dried to remove unwanted volatile processing solvent and provide a drug-in-adhesive (DIA) matrix, which is then brought into contact with a second support, such as the backing layer, in any manner known to those of skill in the art. Such techniques include calender coating, hot melt coating, solution coating, etc.

An exemplary general method of preparation of single layer, drug-in-adhesive transdermal patches is as follows:

1. Appropriate amounts of water-insoluble, pressure sensitive adhesive, dissolved in an appropriate solvent, is placed in a mixing vessel;

2. The drug is then added, neat or presolubilized in a volatile processing solvent, to the adhesive-solvent mixture and mixing agitation is carried out until the drug is uniformly incorporated therein to provide a drug-in-adhesive (DIA) mixture.

3. Penetration enhancer(s), any optional co-solvent, and excipients, if any, are then added to the DIA mixture, and thoroughly mixed to provide a substantially water-insoluble wet-base formulation.

4. The wet-base formulation is permitted to deaerate, as necessary, and is then transferred to a coating operation where it is uniformly cast or coated onto a release liner at a controlled specified thickness to provide a DIA coating. The wet-base formulation may be cast by pouring the wet-base solution onto the release liner (or other support) preferably to a wet coating thickness in a range of about 400 to about 1000 μm, more preferably in a range of about 500 to about 900 μm, and most preferably in a range of about 600 to about 800 μm.

5. The DIA coating is then substantially dried, such as by passing the coated product through an oven, to drive off all unwanted volatile processing solvents to provide a substantially dried DIA matrix.

6. The substantially dried DIA matrix on the release liner is then laminated to the backing material by an conventional laminating technique, such as rolling under uniform pressure, and wound into rolls or folded into sheets for storage or further manufacture of patches.

7. Appropriately sized and shaped individual dosage units may be die-cut from the roll or sheet material and then packaged, such as in a heat sealed pouch or packet.

A therapeutic dose of antiemetic can be delivered to a mammal, preferably a human, by removing the liner from a dosage unit of the transdermal antiemesis delivery system, contacting the skin surface with the skin adhesive DIA matrix and maintaining the contact for a period sufficient to maintain effective antiemetic therapy. The foregoing therapeutic method preferably is practiced by effecting skin contact at least once daily. A four-day or twice-weekly dosage regimen is preferred.

A presently preferred transdermal patch embodiment for a four-day or twice weekly dosage interval has an active dosage area size in the range of about 10 to about 20 cm², has a drug loading in the range of about 10 to about 15 mg/patch; is prepared with a wet-base skin adhesive composition wet coating thickness in the range of about 600 to about 800 μm; has a dry coating weight in the range of about 100 to 400 mg/patch, preferably in the range of about 150 to about 300 mg/patch; maintains a drug flux in the range of at least about 6 to about 12 μg/cm²/hour; has no visibly observable drug crystals in the skin adhesive coating over a period of at least 4 days storage at an ambient temperature of about 25° C. and relative humidity in the range of about 20%, not more than about 60%, or at least 14 days storage at an ambient temperature of about 40° C. and relative humidity of about 75%; has a backing film material having an MVTR value of not more than about 200 g/m²/24 hours, preferably not more than about 20 g/m²/24 hours; and a release liner. A particularly preferred release liner is a silicone-coated PET or fluoropolymer coated polyester. A particularly preferred backing film material is a polyester/EVA laminate (e.g., SCOTCHPAK™ 9732). Preferably the transdermal patch has a tack adhesion peak force of at least about 3 Newtons (N) per square centimeter (cm²).

The transdermal antiemesis delivery system may be provided for antiemesis therapy in kit form with instructional indicia included therein for use.

Instructional indicia includes, without limitation, printed media, aural media, visual aids, electronic media or a combination thereof which inform and instruct the user. Printed media include, but are not limited to, labels, pamphlets, books, flyers and the like. Aural media include, but are not limited to, tape recordings, audio compact disks, records, and the like. Visual aids include, but are not limited, to photographs, slides, movies, videos, DVDs, and the like. Electronic media include all forms of electronic data storage media, such as, but not limited to, diskettes, interactive CD-ROMs, interactive DVDs, and the like.

The following examples further illustrate the preparation and use of preferred embodiments but are not intended to be limiting.

Methods:

Unless indicated otherwise in the Examples, the following methods were used.

I. Skin Permeation

Skin permeation was performed in vitro, using intact human cadaver skin mounted on a static diffusion cell (Franz-cell type) having side-by-side permeation cells (PermeGear Horizontal diffusion cell, PermeGear, Inc.). The volume capacity of each of the donor cell and of the receptor cell was about 5.2 ml, and the permeation area was about 0.64 cm².

The human cadaver skin was stored in a deep freezer at a temperature of about −70° C., then thawed for use and immersed in aqueous 0.9% sodium chloride solution. For testing, the thawed skin was cut into squares of a dimensional size of about 2 cm×2 cm, and the test patch was also cut into squares of dimensional size of about 2 cm×2 cm. The release liner was removed from the test patch, the surface of the skin adhesive side of the patch was applied to the epidermal side of a square of skin to provide a patch/skin composite. The patch/skin composite was mounted on each half cell of the diffusion cell as a permeable membrane. A film of PET/EVA laminate was placed between each patch/skin composition, and the half cells were then placed together.

The medium used in the donor cell and in the receptor cell was 70 mM pH 7.4 phosphate buffered saline (0.85% sodium chloride). About 3 to about 5 milliliters (ml) of the medium was added to each cell. A magnetic stirring rod was placed in each cell and the medium was stirred at a rate of about 600 revolutions per minute and the temperature of the medium was maintained at a temperature of about 32° C. throughout the test period. A sample of about 0.15 to about 0.2 ml permeate was manually collected at each sampling time over a period of about 24 or 96 hours, and assayed for granisetron using High Performance Liquid Chromatography (HPLC) technique. A Franz cell equipped with an auto sampling system can, of course, also be used.

The HPLC assay was performed with an HPLC column (XTerra RP18, Waters) having dimensions of 4.6 mm by 150 mm, and a phenyl 5 micron packing. A photodiode array detector was used to detect the drug peak that eluted at about 5 to about 6 minutes. The flow rate (ml/min), buffer pH, the mobile phase composition, and wavelength (nm) at which the data was detected for assay analysis is shown in Table A for HPLC methods A-E referred to in the following Examples.

TABLE A

| HPLC | Flow Rate | Buffer¹ | Composition | Analytical |
|---|---|---|---|---|
| Method | (ml/min) | pH | of Mobile Phase | Wavelength (nm) |
| A | 0.75 | 5.2 | 60% Acetate Buffer¹ | 254 |
| | | | 40% Acetonitrile | |
| B | 0.75 | 10 | 10% Ammonium | 280 |
| | | | Bicarbonate Buffer² | |
| | | | 50% Acetonitrile | |
| | | | 40% Water | |
| C | 0.75 | 10 | 10% Ammonium | 300 |
| | | | Bicarbonate Buffer² | |
| | | | 50% Acetonitrile | |
| | | | 40% Water | |
| D | 0.75 | 5.2 | 65% Acetate Buffer¹ | 280 |
| | | | 20% Acetonitrile | |
| | | | 10% Methanol | |
| | | | 5% Water | |
| E | 0.15 | <3.1 | 90% (0.1%) | 230 |
| | | | Phosphoric Acid | |
| | | | 10% Acetonitrile | |

Notes to Table A:
¹60 mM sodium acetate.
²100 mM ammonium bicarbonate.

II. Drug Release

The drug release, in micrograms per square centimeter per hour, (reported as either μg/cm²/hr. or μg/cm²/hr.^{1/2}) or in % over a period of about 24 hours from the patches was determined by dissolution technique using the USP rotating paddle Apparatus-IV (or V, where indicated) generally described in United States Pharmacopeia XXIII, (USP), Drug Release Physical Tests, Chapter 724, Apparatus IV, p. 2018. For use in drug release tests, the patches were sized to about 9 to about 10 cm², the release liner was removed and the patch was mounted in the Apparatus. The test medium was about 500 milliliters of a 70 mM phosphate buffered saline solution. The paddle speed was set at about 50 revolutions per minute (RPM) and the operating temperature was set at about 32° C. At each sample time, about one milliliter (ml) of sample was withdrawn and the granisetron content was assayed by the HPLC technique previously described. The mean values of three runs per patch were determined.

III. Tack Testing

Tack adhesion was determined using standard adhesion ASTM Probe Tack Tester techniques. The release liner was removed from the patch, the patch was mounted on the tester, and peak removal force was measured in Newtons (N), with a 0.5 mm² probe. Patches were dimensioned as to be 2 cm×2 cm square. A tack adhesion peak force value of at least about 0.5 N/cm², and more preferably greater than about 3 N/cm² to at least greater than about 15 N/cm², are judged acceptable.

IV. Preparation of Compositions and Patches

Unless otherwise indicated, the compositions were prepared by solubilizing granisetron base in penetration enhancer, co-solvent and adhesive and mixing together by stirring slowly until all ingredients dissolved, for a period of several hours or until homogeneous to provide a drug-in-adhesive wet-base solution. The wet-base solution was allowed to de-aerate on standing.

Patches were prepared by pouring the wet-base solution onto a silicone coated polyester release liner, (MEDIRELEASE® 2249, Mylan Technologies, Inc., or silicone coated PET Liner Grade 24950, Loparex, Inc.), or a fluoropolymer coated release liner (SCOTCHPAK™ 1022, 3M), as indicated in the Examples, to provide a cast (wet-base) film having a thickness in the range of about 400 to about 1,000 micrometers (em) or as specifically indicated in the Examples below. The terms “wet-base” or “pour thickness” are used for convenience. The cast film was then substantially dried in a laboratory dryer unit to volatilize substantially all unwanted solvents.

A backing material was then laminated to the substantially dried DIA matrix by rolling the backing material onto the DIA matrix using substantially uniform pressure. For use, patches were die cut to the desired dimensions. For “occlusive” type transdermal systems, a backing film of SCOTCHPAK™ 9732, 3M, polyester/EVA laminate, was then laminated onto the substantially dried drug-in-adhesive (DIA) coated surface.

Unless otherwise indicated, transdermal antiemesis patches were dimensioned to be about 10 to about 20 cm² for use in the tests as indicated in the Examples below.

### EXAMPLE 1

This example illustrates transdermal skin adhesive compositions and patches containing varying amounts of granisetron, varying amounts of penetration enhancers and water-insoluble pressure sensitive adhesive in the drug-in-adhesive (DIA) matrix, in the amounts indicated in Table 1.

The compositions and patches were prepared generally by the procedure of Method IV, except that both the drug load and permeation enhancer content in the skin adhesive composition were varied, as shown in Table 1. The total granisetron base content, based on the weight of the dry DIA matrix of the transdermal patch, was varied in the range of about 4.5 to about 9 weight %. The total amount of penetration enhancer in the skin adhesive composition was varied from zero to about 40 weight %, based on the weight of the dry DIA matrix of the transdermal patch, and the remainder of the DIA matrix composition was made up of a commercial polyacrylate adhesive (GELVA® 3083).

TABLE 1

| | | Penetration | Penetration | |
|---|---|---|---|---|
| Patch | Granisetron | Enhancer (1) | Enhancer (2) | PAC |
| Example | Weight % | Weight % | Weight % | Adhesive |
| 1-A | 4.5 | DGME 5% | NMP 5% | Q.S. to100% |
| 1-B | 4.5 | DGME 5% | None | Q.S. to100% |
| 1-C | 4.5 | NMP 5% | None | Q.S. to100% |
| 1-D | 4.5 | DGME 10% | NMP 10% | Q.S. to100% |
| 1-E | 4.5 | DGME 5% | DDAIP 5% | Q.S. to100% |
| 1-F | 4.5 | NMP 5% | DDAIP 5% | Q.S. to100% |
| 1-G | 9 | DGME 10% | NMP 10% | Q.S. to100% |
| 1-H | 4.5 | DGME 40% | None | Q.S. to100% |
| 1-I | 4.5 | DGME 20% | NMP 20% | Q.S. to 100% |
| 1-J | 4.5 | None | NMP 40% | Q.S. to 100% |
| 1-K | 4.5 | None | None | Q.S. to 100% |
| 1-L | 4.5 | DGME 10% | SPAN ® 20 | Q.S. to 100% |
| | | | 10% | |

Notes to Table 1:
PAC: Polyacrylate adhesive, (GELVA ® 3083).
NMP: N-Methyl-2-pyrrolidone
DGME: Diethylene glycol monoethyl ether
DDAIP: Dodecyl-2-N,N-dimethylaminopropionate (base form)
Q.S. = Quantity sufficient.

Transdermal patches were prepared with each of the compositions in Table 1 (A-L), using a fluoropolymer coated polyester release liner (SCOTCHPAK™ 1022) and a wet-base coat film thickness in the range of about 100 to about 400 micrometers (μm). The % drug load content of the substantially dry DIA matrix is shown in Table 1 along with the estimated % amount of total penetration enhancer. An occlusive backing film (SCOTCHPAK™ 9732, 3M, polyester/EVA laminate) was laminated onto the substantially dried drug-in-skin adhesive matrix.

No crystals were observable initially in the DIA matrix of any of the transdermal patches, based on microscopic examination, but after a storage period of about 12 hours, some granisetron crystals were observed in the transdermal patch of Example 1-A.

### EXAMPLE 2

This example illustrates the skin permeation of granisetron through human cadaver skin, in vitro, from each of the patches of Example 1 (A-L).

Skin permeation was performed in vitro using human cadaver skin mounted on a Franz cell, having side-by-side skin permeation cells as described in Skin Permeation Method I, except that the medium used in the receptor cell was 50 mM pH 7.4 phosphate buffered saline (0.85% sodium chloride). For testing, the skin was cut into squares of a dimensional size of about 2 cm×2 cm and each transdermal patch of Example 1 (A-L) was cut to a dimensional size of about 2 cm×2 cm square. The skin permeation rate in micrograms/square centimeters/hour (μg/cm²/hr.) was determined by Method I using HPLC Method E (Table A). The skin permeation rate, the lag time in hours and % skin permeation relative to the skin permeation of the transdermal patch of Example 1-A are shown in Table 2 for three studies (Study 1, Study 2 and Study 3).

TABLE 2

| | | | | | Relative |
|---|---|---|---|---|---|
| Patch | Skin Permeation Rate¹ | | | Lag Time | Skin |
| Example | STUDY 1 | STUDY 2 | STUDY 3 | (hr.) | Permeation² |
| 1-A | 3.737 | 5.544 | 13.84 | 3.21 | 100 |
| | | | | to 6.23 | |
| 1-B | 4.456 | — | — | 6.09 | 119 |
| 1-C | 5.057 | — | — | 5.09 | 135 |
| 1-D | 5.370 | — | — | 2.96 | 144 |
| 1-D | 6.533 | — | — | to 6.04 | 175 |
| 1-E | — | — | 18.58 | 5.05 | 134 |
| 1-F | — | — | 18.12 | 4.92 | 131 |
| 1-G | 3.148 | — | — | 3.03 | 80 |
| 1-H | — | 6.271 | — | 4.45 | 113 |
| 1-I | — | 5.454 | — | 4.10 | 98 |
| 1-J | — | 6.158 | — | 4.12 | 111 |
| 1-K | — | — | 12.493 | 2.32 | 90 |
| 1-L | — | — | 18.443 | 2.61 | 133 |

Notes to Table 2:
¹Unit: μg/cm²/hr.
²Skin permeation rate relative to Example 1-A, in the study indicated.

The data showed that the skin permeation rate of granisetron base through human cadaver skin from the transdermal patches was in the range of about 3 to about 20 μg/cm²/hr. with a lag time in the range of about 2 to about 6.5 hours.

### EXAMPLE 3

The incorporation of granisetron base in various penetration enhancers and solvents was determined by incrementally adding small amounts of the drug to about 100 ml of liquid penetration enhancer, and agitating the liquid mixture until the drug was no longer incorporated (i.e., saturation) to provide an apparently saturated liquid mixture at an ambient room temperature in the range of about 20 to about 25° C. Each of the saturated liquid mixtures was agitated by inverting for about 24 hours at ambient room temperature, the solution was centrifuged and its supernatant was diluted in HPLC mobile phase and assayed by the HPLC method as described in Example 2.

The solubility of granisetron base determined in micrograms/ml of permeation enhancer is shown in Table 3.

TABLE 3

| | Granisetron Solubility* |
|---|---|
| Penetration Enhancer | (μg/ml.) |
| PEG-400 | 45.71 |
| Propylene glycol | 31.71 |
| BRIJ ® 30 | 41.24 |
| SPAN ® 20 | 32.94 |
| Isopropyl myristate | 5.46 |
| NMP | 196.39 |
| DGME (transcutol) | 86.6 |
| DDAIP (free base) | 7.17 |
| Limonene | 8.59 |

Notes to Table 3:
*At ambient room temperature in the range of about 20 to about 25° C.
NMP: N-methyl-2-pyrrolidone
DGME: Diethylene glycol monoethyl ether
DDAIP: Dodecyl-2-N,N-dimethylaminopropionate

### EXAMPLE 4

This example illustrates the skin permeation of granisetron base from a liquid medium containing granisetron base in combination with N-methyl-2-pyrrolidone (NMP), used as the sole skin permeation enhancer (Ex. 4-A), or in further combination with one of the following skin permeation enhancers, diethylene glycol monoethyl ether (DDAIP) (Ex. 4-B), triacetin (Ex. 4-C), isosorbide dimethyl ether (DMI) (Ex. 4-D), sorbitan monolaurate (SPAN® 20) (Ex. 4-E), or triethylcitrate (TEC) (Ex. 4-F). The combinations were generally prepared by the procedure of Method IV, in the calculated % amounts shown in Table 4.

TABLE 4

| | Granisetron | DDAIP | Triacetin | DMI | SPAN ® 20 | TEC | NMP |
|---|---|---|---|---|---|---|---|
| Ex. | Wt. % | Wt. % | Wt. % | Wt. % | Wt. % | Wt. % | Wt. % |
| 4-A | 5.12 | — | — | — | — | — | 94.88 |
| 4-B | 4.98 | 4.95 | — | — | — | — | 90.07 |
| 4-C | 5 | — | 5.16 | — | — | — | 89.84 |
| 4-D | 4.97 | — | — | 5.35 | — | — | 89.68 |
| 4-E | 4.98 | — | — | — | 4.95 | — | 90.07 |
| 4-F | 5 | — | — | — | — | 5.33 | 89.67 |

Skin permeation was evaluated in vitro using human cadaver skin as the membrane and a flow-through Franz cell. The procedure of the Skin Permeation Method I was used, except that test solution, in an amount of about 500 μl, was placed directly onto the epidermal side of the human cadaver skin mounted on the permeation cell and samples of permeate were taken every four hours over a period of about 24 hours. The data for skin permeation rate, determined as described in Method I, HPLC Method E (Table A), and lag time, from each of the test solutions of Exs. 4 (A-F) is shown in Table 4A.

TABLE 4A

| | | Skin | | |
|---|---|---|---|---|
| | Permeation | Permeation | | |
| Ex. | Enhancer(s) | Rate¹ | S.E.² | Lag time (hr.) |
| 4-A | NMP | 15.32 | 1.54 | 5.33 |
| 4-B | NMP/DDAIP | 74.19 | 5.64 | 5.10 |
| 4-C | NMP/Triacetin | 15.79 | 1.04 | 5.20 |
| 4-D | NMP/DMI | 23.52 | 0.64 | 6.76 |
| 4-E | NMP/SPAN ® 20 | 61.77 | 5.58 | 5.66 |
| 4-F | NMP/TEC | 14.39 | 0.28 | 6.77 |

Notes to Table 4A:
¹Unit: μg/cm²/hr
²S.E. = Standard Error.

The data show that the highest skin permeation rate was achieved from a combination of NMP and DDAIP (Ex. 4-B) and a combination of NMP and SPAN® 20 (Ex. 4-E).

### EXAMPLE 5

This example illustrates embodiments of transdermal drug-in-adhesive matrix patches containing granisetron base, N-methyl-2-pyrrolidone (NMP), as the sole permeation enhancer (Exs. 5-(A-F), and 5-N), or in further combination with one or more of the following permeation enhancers: sorbitan monolaurate (SPAN® 20) (Exs. 5-G, 5-I, 5-K, 5-M, 5-O and 5-Q), sorbitan monooleate (CRILL™ 4NF), (Exs. 5-H, and 5-J) and diethylene glycol monoethylether (DGME) (Exs. 5-L, 5-M, 5-P and 5-Q) prepared with various acrylic-based adhesives identified, and in the amounts indicated, based on the weight of the substantially dry drug-in-adhesive (DIA) matrix of the transdermal patch, shown in Table 5.

TABLE 5

| Patch | Granisetron | NMP | SPAN ® 20 | CRILL ™ 4 | DGME | Adhesive |
|---|---|---|---|---|---|---|
| Ex. | Base Wt. % | Wt. % | Wt. % | Wt. % | Wt. % | Q.S. to 100% |
| 5-A | 6 | 5 | — | — | — | A |
| 5-B | 6 | 5 | — | — | — | B |
| 5-C | 6 | 5 | — | — | — | C |
| 5-D | 6 | 5 | — | — | — | D |
| 5-E | 6 | 5 | — | — | — | E |
| 5-F | 6 | 5 | — | — | — | B |
| 5-G | 6 | 5 | 10 | — | — | B |
| 5-H | 6 | 5 | — | 10 | — | B |
| 5-I | 6 | 5 | 10 | — | — | E |
| 5-J | 6 | 5 | — | 10 | — | E |
| 5-K | 6 | 5 | 5 | — | — | E |
| 5-L | 6 | 5 | — | — | 10 | E |
| 5-M | 6 | 5 | 5 | — | 10 | E |
| 5-N | 6 | 5 | — | — | — | E |
| 5-O | 6 | 5 | 5 | — | — | B |
| 5-P | 6 | 5 | — | — | 5 | B |
| 5-Q | 6 | 5 | 5 | — | 5 | B |

Notes to Table 5:
Adhesive A = DURO-TAK ® 87-2353
Adhesive B = GELVA ® 3083
Adhesive C = DURO-TAK ® 87-2194
Adhesive D = GELVA ® 2873
Adhesive E = DURO-TAK ® 87-2516

The drug-in-adhesive compositions were prepared by the general Method IV. Transdermal patch Exs. 5-(A-E) were prepared with each of the compositions in Table 5 using a wet-base coat having a film thickness of about 400 micrometers (μm), and patch Exs. 5-(F-Q) were prepared from a wet-base cast of about 600 μm. The preferred release liner used for the patch compositions prepared with Adhesive E (DURO-TAK® 87-2516) was silicone-coated polyester (Loparex PET Liner Grade 24950) and the preferred release liner used for the patch compositions prepared with Adhesives A, B, C and D was fluoropolymer coated polyester (SCOTCHPAK™ 1022).

The % drug load content of the substantially dried DIA matrix is shown in Table 5 along with the estimated % amount of total penetration enhancer. An occlusive backing film (SCOTCHPAK™ 9732, 3M, polyester/EVA laminate) was then laminated onto the substantially dry DIA matrix. The laminate was then stored and subsequently die-cut into patches having a dimensional size of about 10 to about 20 cm².

No crystals were visually observable in the DIA matrix of the transdermal patches stored over a period of about 12 hours at an ambient room temperature in the range of 20° C. to about 25° C. and ambient relative humidity in the range of about 40% to about 60%.

After a storage period of about 3 days under the foregoing ambient atmospheric conditions, some relatively small, sporadic crystalline needles were visible on the surface of patch Exs. 5-B, 5-F, 5-G and 5-H and extensive crystallization was visible in patch Ex. 5-O, all prepared with Adhesive B (GELVA® 3083). After a storage period of about 3 days under the foregoing ambient atmospheric conditions, some relatively small, sporadic crystalline needles were visible on the surface of patch Exs. 5-E, 5-I, 5-K and 5-N, prepared with Adhesive E (DURO-TAK® 87-2516). Under the same foregoing storage conditions, no crystals were visible in patch examples. prepared with Adhesive A (Ex. 5-A), Adhesive C (Ex. 5-C), Adhesive D (Ex. 5-D), or patch Ex. 5-J, prepared with Adhesive E. Regardless of whether Adhesive B or E was used, extensive crystallization was visible in the patches containing DGME permeation enhancer; i.e., patch Exs. 5-L, 5-M, 5-P and 5-Q.

### EXAMPLE 6

This example illustrates the skin permeation of granisetron through human cadaver skin, in vitro, from the transdermal patches of Example 5 (A-E, and K-M), prepared as described in Example 5 and the % drug release in vitro from the transdermal patches of Exs. 5-B, 5-C, 5-D and 5-E. The transdermal patch Exs. 5 (A-E) were cast from a wet-base coating thickness of about 400 μm, and the transdermal patch Exs. 5 (K-M) were cast from a wet-base coating thickness of about 600 μm.

Skin permeation was performed in vitro using human cadaver skin mounted on a Franz cell having side-by-side permeation cells as described in Skin Permeation Method I. For testing, the skin was cut into squares of a dimensional size of about 2 cm×2 cm, and each transdermal patch of Example 5-(A-E, and K-M) was cut to a dimensional size of about 2 cm×2 cm square. The skin permeation rate in μg/cm²/hour, together with the lag time in hours, and granisetron assay in mg/10 cm² patch, are shown in Table 6.

TABLE 6

| | Skin | | | |
|---|---|---|---|---|
| | Permeation | | | |
| Transdermal | Rate | Lag Time | Assay | Wet Base |
| Patch Ex. | (μg/cm²/hr.) | (Hour) | (mg/10 cm²) | Thickness |
| 5-A | 0.624 | — | 5.5-5.86 | 400 μm |
| 5-B | 9.22 | 1.25 | 5.28-6.16 | |
| 5-C | 0.225 | — | 6.12-8.07 | |
| 5-D | 0.24 | — | 4.47-4.47 | |
| 5-E | 12.1-13.67* | 1.32-1.5 | 6.08-6.12 | |
| 5-K | 22.5 | 0.7 | — | 600 μm |
| 5-L | 11.4 | 0.6 | — | |
| 5-M | 22.3 | 1 | — | |

Notes to Table 6:
*Duplicate tests.

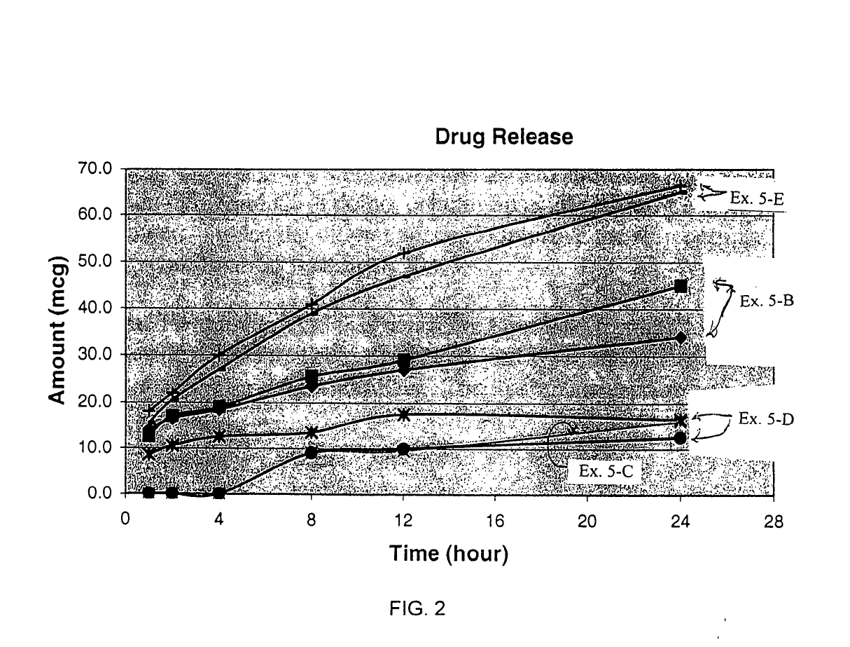

The % drug release was determined over a period of about 24 hours from each of the transdermal patches of Exs. 5-B, 5-C, 5-D, and 5-E as described in the Drug Release Method II. The % drug release results for patch Exs. 5-B, 5-C, 5-D, and 5-E are also graphically shown in FIG. 2 as micrograms granisetron released over a period of 24 hours.

The data show that the drug release was optimal from the transdermal patch of Example 5-E, prepared with Adhesive E (DURO-TAK® 87-2516), followed by the transdermal patch from Example 5-B, prepared with Adhesive B (GELVA® 3083), compared to the transdermal patch of Example 5-D, prepared with Adhesive D (GELVA® 2873). The % drug release from the transdermal patchof Ex. 5-C, prepared with Adhesive C (DURO-TAK® 87-2194), was generally comparable to that of the transdermal patch Example 5-D.

The DURO-TAK® 87-2516 adhesive, which is characterized by the manufacturer as a high shear, low tack, polyacrylate adhesive reportedly having an a hydroxy functional group, was preferred and judged suitable for use in multiple day dosage transdermal patches. Also judged suitable was the GELVA® 3083 adhesive, which is characterized by the manufacturer as a low shear, high tack polyacrylate adhesive reportedly having no functional monomer group. GELVA® 3083 can be used alone or in combination with a high shear, polyacrylate adhesive grade, such as GELVA® 3235.

### EXAMPLE 7

This example illustrates the % skin permeation of granisetron through human cadaver skin, in vitro, determined as described in Skin Permeation Method I, from transdermal patch Exs. 5-F, 5-G, 5-H, 5-I, 5-J, and 5-N prepared as described in Example 5 and cast from a wet-base thickness of about 600 μm. The drug assays were performed as described in Method I, HPLC Method A (Exs. 5-(F-J), and 5-N)); HPLC Method B (Exs. 5-G); and HPLC Method D (at 254 nm) (Exs. 5-(F-J) and 5-N)).

The data show that the % skin permeation rate achieved after about 10 hours where the permeation enhancer included sorbitan monolaurate (SPAN® 20) (Exs. 5-G and 5-I) was higher by a factor of at least 2 compared to that achieved where the permeation enhancer included sorbitan monooleate (CRILL™ 4) (Exs. 5-H and 5-J) or NMP alone (Ex. 5-N). For example, the % skin permeation rate from the transdermal patches of Exs. 5-G and 5-I was about 20 μg/cm² at about 15 hours and in excess of 40 μg/cm² at about 24 hours, whereas that from the transdermal patches of Exs. 5-H, 5-J, and 5-N was no more than about 10 μg/cm² at about 15 hours and no more than about 20 μg/cm² at about 24 hours. The % skin permeation rate achieved in similar tests performed with the transdermal patches of Ex. 5-L and 5-M were judged substantially comparable to one another. The % skin permeation rate from transdermal patch Ex. 5-F, containing NMP in Adhesive “B” (GELVA® 3083) was less than 5 μg/cm² over 24 hours and therefore judged unacceptably slow. Except for the transdermal patch Ex. 5-F, all the tested patches were judged to have acceptable skin permeation rates.

A preferred transdermal patch embodiment has an active dosage area in the range of about 10 to about 20 cm², a drug loading in the range of about 10 to about 15 mg/patch, is prepared with a wet-base thickness coating in the range of about 600 to about 800 μm.

### EXAMPLE 8

This example illustrates embodiments of transdermal drug-in-adhesive matrix patches containing varying amounts of granisetron base, and as permeation enhancers, a combination of N-methyl-2-pyrrolidone (NMP), and sorbitan monolaurate, (Patch Exs. 8-(A-N), prepared with various acrylic-based adhesives and blends thereof identified, and in the amounts indicated, in Table 7, based on the weight of the substantially dry drug-in-adhesive (DIA) matrix of the transdermal patch. Adhesive B is GELVA® 3083; Adhesive E is DURO-TAK® 87-2516; Adhesive F is GELVA® 3067 and Adhesive G is GELVA® 3235.

TABLE 7

| Patch | Granisetron | NMP | Sorbitan | Adhesive Q.S. |
|---|---|---|---|---|
| Ex. | Base Wt. % | Wt. % | Monolaurate* Wt. % | to 100% |
| 8-A | 6 | 5 | 10 | F |
| 8-B | 6 | 5 | 10 | G |
| 8-C | 6 | 5 | 10 | F/G¹ |
| 8-D | 6 | 5 | 10 | F/G² |
| 8-E | 6 | 5 | 10 | F/G³ |
| 8-F | 6 | 5 | 10 | B/G⁴ |
| 8-G | 7 | 5 | 2.5 | E |
| 8-H | 7 | 5 | 7.5 | E |
| 8-I | 5 | 5 | 2.5 | E |
| 8-J | 5 | 5 | 7.5 | E |
| 8-K | 6 | 5 | — | E |
| 8-L | 6 | 10 | 5 | E |
| 8-M | 5 | 5 | 5 | E |
| 8-N | 5 | 10 | 5 | E |

Notes to Table 7:
*Source: CRILL ™ 1 NF (Croda) used in 8-(A-F); SPAN ® 20 Pharma (Uniqema) used in 8-(G-N).
¹Weight Ratio of F:G = 1:3
²Weight Ratio of F:G = 3:1
³Weight Ratio of F:G = 1:1
⁴Weight Ratio of B:G = 1:1

The drug-in-adhesive compositions were prepared generally by the Method IV. Transdermal patches were prepared with each of the compositions in Table 7 using a wet-base cast film thickness of about 600 μm. The release liner used for patches prepared with Adhesives B, F and G was a silicone coated polyester (Loparex PET Liner Grade 24950), and for patches prepared with Adhesive E was fluoropolymer coated polyester (SCOTCHPAK™ 1022, 3M).

The cast film was then substantially dried as described in Method IV, utilizing a laboratory dryer to provide a substantially dry DIA matrix. The % drug load content of the substantially dried DIA matrix also is shown in Table 7 along with the estimated % amount of total penetration enhancer. An occlusive backing film (SCOTCHPAK™ 9732, 3M, polyester/EVA laminate) was laminated onto the substantially dry DIA matrix.

A wet-base coating thickness of about 600 μm provided an effective drug load of about 8 to about 15 mg/20 cm² based on the weight of the substantially dry DIA matrix on a patch size of about 10 to about 20 cm². No crystals were visually observable in the DIA matrix of the transdermal patches over a period of about 12 hours storage at an ambient room temperature in the range of about 20° C. to about 25° C. and ambient room humidity in the range of about 40% to about 60%.

After a storage period of about 3 days under the foregoing atmospheric conditions, no crystals were observed on patch Exs. 8-J and 8-K; some relatively small, sporadic crystalline needles were visible on the surface of patch Exs. 8-A, 8-B (edges only), 8-(C-F), and 8-(H-I and L-N); and extensive crystallization was visible on patch Ex. 8-D.

The results indicated that patch stability (i.e., lack of crystallization) can be controlled or minimized by appropriately adjusting the drug content, permeation enhancer and adhesive.

Additionally, crystallization was minimized by preferably controlling the ambient relative humidity, as illustrated by subjecting patches of Ex. 8-N, in controlled humidity chambers, to an ambient relative humidity ranging from zero, about 20%, about 40%, about 60%, and about 100% at an ambient temperature of about 25° C. for a period of about 8 days. The results are shown in Table 7A for days 1-4, 7 and 8.

TABLE 7A

| RH | DAY 1 | DAY 2 | DAY 3 | DAY 4 | DAY 7 | DAY 8 |
|---|---|---|---|---|---|---|
| 0% | N | N | N | N | N | N |
| 20% | N | N | N | N | S | S |
| 40% | N | N | N | S | C | C |
| 60% | N | N | S | C | C | C |
| 100% | N | S | C | C | C | C |

Notes to Table 7A:
RH = Relative Humidity at 25° C.
N = No Crystals, S = Small, microscopic crystals, C = visible crystals.

The results show that crystal formation in the DIA was beneficially obviated or minimized by maintaining relatively low humidity.

### EXAMPLE 9

This example illustrates the skin permeation rate and relative drug release rate of granisetron from the transdermal patches of Exs. 8-(A-F), compared to transdermal patches of Exs. 5-G and 5-K (prepared as described in Example 5, and cast from a wet-base coating thickness of about 600 μm).

Skin permeation was performed in vitro, using human cadaver skin mounted on a Franz cell in side-by-side permeation cells as described in the Skin Permeation Method I using HPLC Method D. The skin permeation rate in μg/cm²/hour together with the relative skin permeation rate, based on two separate studies are each shown in Table 8.

The drug release over a period of about 24 hours from each of the transdermal patches of Exs. 5-G and 8-(A-F) was determined, relative to that of patch Ex. 5-K, by the standard dissolution technique described in Drug Release Method II. The drug release is expressed in μg/cm² Square-Root-of-Time-Hour (hour^{1/2}) and the calculated % drug release, relative to that of patch Ex. 5-K, are each shown in Table 8.

TABLE 8

| | Drug | | | | Relative |
|---|---|---|---|---|---|
| | Release | Relative | Skin Permeation | | Skin |
| | Rate | Drug Release | Rate³ | | Permeation |
| Patch | (μg/cm²/ | Rate¹ | (μg/cm²/hour) | | Rate¹ |
| Ex. | hour^{1/2}) | (%) | STUDY 1 | STUDY 2 | (%) |
| 8-A | 166.7 | 125.2 | 15.1 | — | 118.9 |
| 8-B | 51.2 | 38.4 | 6.7 | — | 52.8 |
| 8-C | 80.8 | 60.7 | — | 10.3 | 65.6 |
| 8-D | 163.0 | 122.4 | — | 15.8 | 100.6 |
| 8-E | 105.7 | 79.4 | — | 14.0 | 89.2 |
| 8-F | 124.5 | 93.5 | — | 15.6 | 99.4 |
| 5-G | 140.6 | 105.6 | 16.4 | — | 129.1 |
| 5-K | 133.2 | 100 | 12.7 | 15.7 | 100 |

Notes to Table 8:
¹Relative to Patch Ex. 5-K of study indicated.
²Skin permeation rate was conducted in two different studies, Study 1 and Study 2; Patch Ex. 5-K was the reference in each study.

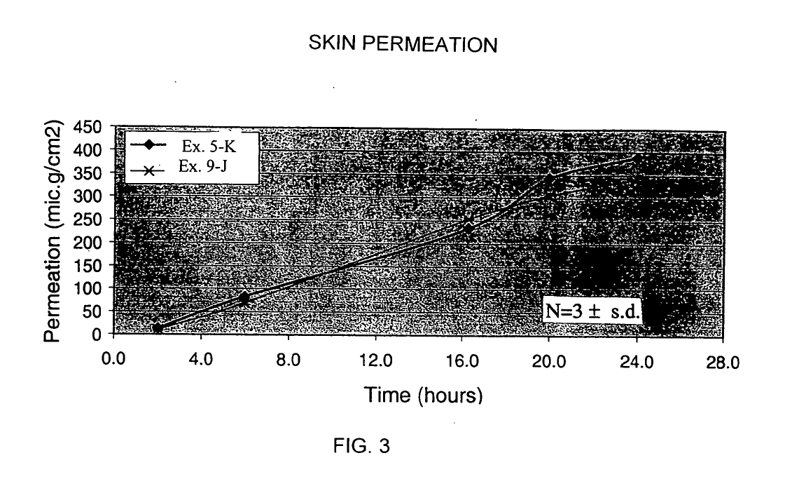

Based on the slopes of the plotted data for drug release rate and skin permeation rate, the skin permeation rate was judged as controlled by the rate of drug release, with differences in lag time attributed to the time taken to saturate the skin during skin permeation. The skin permeation rate of transdermal patch Ex. 5-K compared to that of transdermal patch 8-J, is graphically represented in FIG. 3 in micrograms/cm over a period of about 24 hours.

### EXAMPLE 10

This example illustrates the tack adhesion properties of the transdermal patches of Example 8, patch Exs. 8-(A-F), and of Example 5, patch Exs. 5-G and 5-K. Tack testing was performed as described in Tack Testing Method III. The results for five samples/patch embodiment are shown in Table 9.

TABLE 9
Peak Force Adhesion in Newtons

| Patch | | | | | | | |
|---|---|---|---|---|---|---|---|
| Ex. | 1 | 2 | 3 | 4 | 5 | Avg. | SD |
| 5-G | 1.48 | 1.41 | 1.7 | 1.48 | 1.61 | 1.54 | 0.12 |
| 5-K | 4.02 | 3.87 | 3.88 | 4.59 | 4.23 | 4.12 | 0.30 |
| 8-A | 1.43 | 1.98 | 1.61 | 1.78 | 1.67 | 1.69 | 0.20 |
| 8-B | 1.01 | 0.91 | 0.91 | 0.92 | 0.89 | 0.93 | 0.05 |
| 8-C | 1.05 | 1.06 | 0.87 | 1.13 | 1.25 | 1.07 | 0.14 |
| 8-D | 1.43 | 1.38 | 1.49 | 1.56 | 1.64 | 1.50 | 0.10 |
| 8-E | 1.87 | 1.51 | 1.27 | 1.47 | 0.93 | 1.41 | 0.34 |
| 8-F | 1.01 | 0.67 | 0.93 | 0.97 | 0.93 | 0.90 | 0.13 |

Notes to Table 9:
SD = Standard Deviation.

Overall, the tack adhesion of patch Ex. 5-K was preferred.

### EXAMPLE 11

This example illustrates embodiments of drug-in-adhesive (DIA) compositions containing, as permeation enhancers, sorbitan monolaurate, or mineral oil or combination thereof prepared by general Method IV and transdermal patches prepared therefrom. Transdermal patches were prepared using a silicone-coated (PET) release liner (MEDIRELEASE® 2249) and occlusive backing film (SCOTCHPAK™ 9732).

The DIA compositions are shown in Table 10 with the % by weight drug load and % by weight penetration enhancer content of the substantially dried DIA matrix.

TABLE 10

| | | Sorbitan | | |
|---|---|---|---|---|
| | Granisetron | Monolaurate¹ | Mineral Oil | |
| EX. | Base Wt. % | Wt. % | Wt. % | Adhesive² |
| 11-A | 6 | 5 | None | q.s. to 100% |
| 11-B | 6 | None | 5 | q.s. to 100% |
| 11-C | 6 | 5 | 5 | q.s. to 100% |

Notes to Table 10:
¹CRILL ™ 1 NF.
²DURO-TAK ® 87-2516.

The drug release rate in μg/cm²/hour^{1/2}, determined as described in Method II (using USP rotating paddle, Apparatus V) and HPLC Method C (Table A), and lag time, from each of the patches prepared is shown in Table 10A.

TABLE 10A

| | Drug Release | | | |
|---|---|---|---|---|
| Patch | Rate¹ | Standard | Lag Time | |
| Ex. | (μg/cm²/hr.^{1/2}) | Error | (hr.^{1/2}) | R² |
| 11-A | 148.0 | 3.7 | −0.1 | 0.9975 |
| 11-B | 115.2 | 4.3 | 0.1 | 0.9978 |
| 11-C | 145.0 | 3.4 | 0.1 | 0.9944 |

Notes to Table 10A:
R² = Correlation coefficient for linear least squares fit.

The drug release from and physical stability of either Patch Ex. 11-A was judged greater than that of Patch Exs. 11-(B-C).

### EXAMPLE 12

This example illustrates embodiments of drug-in-adhesive (DIA) compositions containing varying amounts of drug and sorbitan monolaurate, alone or in combination with N-methyl-2-pyrrolidone (NMP) or mineral oil in the amounts shown in Table 11 and transdermal matrix patches prepared therefrom. The % by weight drug load and % by weight penetration enhancer amount of the substantially dried DIA matrix is shown in Table 11.

TABLE 11

| | | Sorbitan | | Mineral | |
|---|---|---|---|---|---|
| | Granisetron | Monolaurate¹ | NMP | Oil² | |
| EX. | Base Wt. % | Wt. % | Wt. % | Wt. % | Adhesive³ |
| 12-A | 5 | 7.5 | 5 | None | q.s. to 100% |
| 12-B | 5 | 10 | 5 | None | q.s. to 100% |
| 12-C | 5 | 10 | None | None | q.s. to 100% |
| 12-D | 6 | 10 | 5 | None | q.s. to 100% |
| 12-E | 6 | 10 | None | None | q.s. to 100% |
| 12-F | 5 | 10 | None | None | q.s. to 100% |
| 12-G | 5 | 5 | None | None | q.s. to 100% |
| 12-H | 5 | 7.5 | None | None | q.s. to 100% |
| 12-I | 5 | 5 | None | 5 | q.s. to 100% |
| 12-J | 5 | 5 | 10 | None | q.s. to 100% |

Notes to Table 11:
¹Source: CRILL ™ 1 NF used in 12-(B-E) and 12-(G-I) and SPAN ® 20 Pharma Used in 12-A, 12-F and 12-J.
²Light mineral oil, 34.5 cs min. at 40° C., sp. gr. 0.845-0.905 at 75° C.
³DURO-TAK ® 87-2516.

The drug-in-adhesive compositions were prepared generally by Method IV and transdermal patches were prepared with each of the compositions in Table 11 using a silicone coated PET release liner (MEDIRELEASE® 2249) and occlusive backing polyester/EVA laminate film (SCOTCHPAK™ 9732).

The drug release data in micrograms/cm²/hour^{1/2} determined by Method II, HPLC Method C (Table A), for patches prepared with Exs. 12-B, 12-C, 12-D, and 12-E. The data showed that the drug release rate from Patch Exs. 12-D and 12-E was faster than that from Patch Exs. 12-B and 12-C, and formation of drug crystals in the matrix was also observed sooner. The patches were judged physically stable for about seven days storage at an ambient temperature of about 25° C. and 60% relative humidity. Surprisingly, when the same compositions were prepared and patches made using a silicone-coated PET release liner from a different supplier (Loparex), drug crystals were observed in the matrix within about three days under the foregoing storage conditions.

The skin permeation rate in vitro through human cadaver skin was determined by Method I for Patch Exs. 12-C, 12-G and 12-H over a period of 96 hours, and, as shown in Table 11A, each sustained a granisetron skin permeation rate greater than 8 micrograms/cm²/hour^{1/2}.

TABLE 11A

| | Skin | | | |
|---|---|---|---|---|
| | Permeation | | | |
| Patch | Rate | Standard | Lag Time | |
| Ex. | (μg/cm²/hr.) | Error | (hours) | R² |
| 12-C | 10.29 | 0.77 | −0.06 | 0.9783 |
| 12-G | 8.87 | 0.42 | 1.65 | 0.9910 |
| 12-H | 8.21 | 0.35 | 2.0 | 0.9928 |

Note to Table 11A:
R² = correlation coefficient for linear least squares fit.

Patch Exs. 12-C, 12-G and 12-H were separately stored unpackaged, in a stability test chamber having an ambient temperature of about 40° C. and relative humidity of about 75%, and each one of the embodiments had crystal formation in the DIA matrix within a storage period of about 24 hours. The stability of the compositions was judged to be substantially unaffected by the source of the sorbitan monolaurate used, based on the extent or onset time for drug crystal formation in the DIA matrix observed on storage. For example, the composition and patch of Example 12-F was also successfully prepared with sorbitan monolaurate obtained from a commercial supplier (Aldrich).

### EXAMPLE 13

This example illustrates embodiments of drug-in-adhesive (DIA) compositions containing varying penetration enhancers and amounts and transdermal patches prepared therefrom. Transdermal patches were prepared using a silicone-coated (PET) release liner (MEDIRELEASE® 2249) and occlusive backing film (SCOTCHPAK™ 9732). The penetration enhancers were sorbitan monolaurate, polyvinylpyrrolidone (PVP), propylene glycol, polyethylene glycol 400 (PEG-400) and dimethicone in the amounts shown in Table 12 along with the % by weight drug load in the substantially dried DIA matrix.

TABLE 12
Weight Percent

| | Granisetron | Sorbitan | | | | | |
|---|---|---|---|---|---|---|---|
| Ex. | Base | monolaurate¹ | PVP² | PG³ | PEG-400 | Dimethicone⁴ | Adhesive⁵ |
| 13-A | 6 | 5 | 1 | None | None | None | q.s. to 100 |
| 13-B | 6 | 5 | 2 | None | None | None | q.s. to 100 |
| 13-C | 6 | 5 | 5 | None | None | None | q.s. to 100 |
| 13-D | 6 | 5 | 10 | None | None | None | q.s. to 100 |
| 13-E | 6 | 5 | None | None | None | 5 | q.s. to 100 |
| 13-F | 6 | 5 | None | 5 | None | None | q.s. to 100 |
| 13-G | 6 | 5 | None | None | 5 | None | q.s. to 100 |
| 13-H | 6 | 5 | 5 | 5 | None | None | q.s. to 100 |
| 13-I | 6 | None | 5 | 5 | None | None | q.s. to 100 |
| 13-J | 6 | None | None | 5 | None | None | q.s. to 100 |

Notes to Table 12:
¹CRILL ™ 1NF.
²Polyvinylpyrrolidone, PLASDONE ™ K29/32 NF.
³PG-Propylene glycol.
⁴Dow Medical Fluid D-100.
⁵DURO-TAK ® 87-2516.

### EXAMPLE 14

The drug release rate from patches prepared with the DIA compositions described for Examples 12-I, 13-E. 13-F and 13-G was determined by Method II, HPLC Method C (Table A). The data are shown on Table 13.

TABLE 13

| Patch | Drug Release Rate | Standard | Lag Time | |
|---|---|---|---|---|
| Ex. | (μg/cm²/hr.^{1/2}) | Error | (hours^{1/2}) | R² |
| 12-I | 145.0 | 3.4 | 0.1 | 0.9987 |
| 13-E | 154.2 | 3.0 | −0.1 | 0.9985 |
| 13-F | 148.1 | 0.8 | 0 | 0.9999 |
| 13-G | 164.1 | 2.9 | 0.1 | 0.9988 |

Note to Table 13:
R² = correlation coefficient for linear least square fit.

Transdermal patch Exs. 12-I and 13-F are preferred. Transdermal Patch Exs. 13-E and 13-G had the highest drug release rate, but transdermal patch Ex. 13-E showed evidence of leaching of the dimethicone from the DIA matrix and transdermal Patch Ex. 13-G had relatively low adhesiveness to skin, based on an in vivo wear test using a placebo patch applied to the upper arm of a human subject. In similar tests of placebo patches, the adhesiveness of patch Ex. 12-I containing mineral oil was preferred over that of patch Ex. 13-F.

In one study (Study I), the skin permeation rate was determined by Method I, HPLC Method C (Table A) for patch Ex. 12-I and compared to that of a patch prepared as described in Example 11-A. In a second study (Study II), the skin permeation rate for patch Exs. 13-E, 13-F and 13-G was determined and also compared against the skin permeation rate of patch Ex. 11-A. The results from both studies are shown in Table 13A, along with the calculated % skin permeation relative to that of patch Ex. 11-A.

TABLE 13A

| | Skin Permeation | | Relative Skin | | | |
|---|---|---|---|---|---|---|
| Patch | Rate | Standard | Permeation Rate^{(a)} | Lag Time | | |
| Ex. | (μg/cm²/hr.) | Error | (%) | (hours) | R² | Study |
| 11-A | 6.13 | 0.42 | 100.0 | 6.5 | 0.9906 | I |
| 12-I | 5.83 | 0.16 | 95.2 | 7.8 | 0.9985 | I |
| 11-A | 8.06 | 0.79 | 100.0 | 2.6 | 0.9814 | II |
| 13-E | 11.02 | 1.43 | 136.7 | 3.6 | 0.9676 | II |
| 13-F | 10.65 | 1.43 | 132.1 | 2.2 | 0.9650 | II |
| 13-G | 11.18 | 1.39 | 138.7 | 6.0 | 0.9699 | II |

Notes to Table 13A:
^{(a)}Relative to Patch Ex. 11-A.
R² = correlation coefficient for linear least square fit.

In Study I, transdermal patch Ex. 12-I, which had a lower amount of granisetron base than transdermal patch Ex. 11-A and a combination of penetration enhancers, had a longer lag time and lower skin permeation rate. In Study II, transdermal patch Exs. 13-E, 13-F and 13-G, which had the same amount of granisetron base as Patch Ex. 11-A, each had a higher skin permeation rate relative to transdermal Patch Ex. 11-A. Transdermal Patch Ex. 13-F was preferred.

The drug release rate was also determined for the transdermal patch Exs. 13-(A-D) prepared with DIA compositions containing a combination of PVP and sorbitan monolaurate penetration enhancers. The data surprisingly showed that the drug release rate increased as the amount of PVP present in the DIA matrix increased from 1 to 10% by weight.

The in vitro skin permeation rate was determined by Method I, HPLC Method C (Table A) for transdermal patch Exs. 13-C and 13-D and compared to the skin permeation rate of transdermal patch Ex. 11-A. The results are shown in Table 13B, along with the calculated skin permeation rate relative to that of transdermal patch Ex. 11-A.

TABLE 13B

| | Skin | | Relative Skin | | |
|---|---|---|---|---|---|
| | Permeation | | Permeation | | |
| Patch | Rate | Standard | Rate^{(a)} | Lag Time | |
| Ex. | (μg/cm²/hr.) | Error | (%) | (hours) | R² |
| 11-A | 7.14 | 0.35 | 100.0 | 2.37 | 0.9953 |
| 13-C | 7.72 | 0.55 | 108.2 | 3.15 | 0.9899 |
| 13-D | 5.14 | 0.67 | 72.0 | 3.53 | 0.9672 |

Notes to Table 13B:
^{(a)}Relative to patch Ex. 11-A.
R² = correlation coefficient for linear least square fit.

Transdermal patch Ex. 13-C, which contained about 5% by weight PVP, was judged optimal for increased drug release and skin permeation.

### EXAMPLE 15

This example illustrates embodiments of drug-in-adhesive (DIA) compositions containing varying amounts of drug and varying amounts of combinations of penetration enhancers and transdermal patches prepared therefrom. Transdermal patches were prepared using a silicone-coated (PET) release liner (MEDIRELEASE® 2249) and occlusive backing film (SCOTCHPAK™ 9732). The penetration enhancers were PVP, polyoxyethylene (4) lauryl ether (Laureth-4), propylene glycol, and mineral oil in the amounts shown in Table 14 along with the % by weight drug load in the substantially dried DIA matrix.

TABLE 14
Weight Percent

| | Granisetron | | | | | |
|---|---|---|---|---|---|---|
| Ex. | Base | PVP¹ | Laureth-4² | PG³ | Mineral Oil⁴ | Adhesive⁵ |
| 15-A | 6 | None | 5 | 5 | None | q.s. to 100 |
| 15-B | 5 | 5 | 5 | 5 | None | q.s. to 100 |
| 15-C | 4 | 5 | 5 | 5 | None | q.s. to 100 |
| 15-D | 5 | 5 | 5 | None | None | q.s. to 100 |
| 15-E | 4 | 5 | 5 | None | None | q.s. to 100 |
| 15-F | 4 | 5 | 4 | 5 | None | q.s. to 100 |
| 15-G | 4 | 5 | 2 | 5 | None | q.s. to 100 |
| 15-H | 4 | 5 | 1 | 5 | None | q.s. to 100 |
| 15-I | 4 | 5 | None | 5 | None | q.s. to 100 |
| 15-J | 4 | 5 | 2 | None | 5 | q.s. to 100 |
| 15-K | 4 | None | 2 | 5 | None | q.s. to 100 |

Notes to Table 14:
¹Polyvinylpyrrolidone, PLASDONE ™ K29/32 NF.
²INCI name for polyoxyethylene (4) lauryl ether, BR1J ® 30.
³PG = Propylene glycol
⁴Light mineral oil, 34.5 cs min. at 40° C., sp. gr. 0.845-0.905 at 75° C.
⁵DURO-TAK ® 87-2516

The drug release rate from patches prepared with the DIA compositions of Exs. 15 (A-J) was determined by Method II, HPLC Method C (Table A). The data are shown in Table 14A.

TABLE 14A

| Patch | Drug Release Rate | Standard | Lag Time | |
|---|---|---|---|---|
| Ex. | (μg/cm²/hr.^{1/2}) | Error | (hours^{1/2}) | R² |
| 15-A | 163.4 | 10.5 | 0.6 | 0.9918 |
| 15-B | 176.1 | 7.8 | 0.2 | 0.9923 |
| 15-C | 113.2 | 7.1 | 0.3 | 0.9846 |
| 15-D | 135.3 | 1.7 | 0.1 | 0.9994 |
| 15-E | 111.2 | 3.8 | 0.2 | 0.9954 |
| 15-F | 117.4 | 3.6 | 0.2 | 0.9962 |
| 15-G | 106.2 | 1.4 | 0.1 | 0.9993 |
| 15-H | 106.1 | 1.9 | 0.3 | 0.9998 |
| 15-I | 85.1 | 0.7 | 0.1 | 0.9997 |
| 15-J | 93.3 | 0.7 | 0.1 | 0.9980 |

Note to Table 14A:
R² = correlation coefficient for linear least square fit.

The skin permeation rate was determined for selected patches by Method I, HPLC Method C (Table A) in five separate tests (Studies I-V) compared to that of a patch of Ex. 11-A. In Study I, a patch of Example 13-H was also included for comparison. The results from each study are shown in Table 14B.

TABLE 14B

| Patch | Skin Permeation Rate | Standard | Lag Time | | |
|---|---|---|---|---|---|
| Ex. | (μg/cm²/hr.) | Error | (hours) | R² | Study |
| 11-A | 5.3 | 0.3 | 5.8 | 0.9969 | I |
| 13-H | 7.0 | 0.3 | 5.7 | 0.9983 | I |
| 15-A | 13.0 | 0.0 | 5.0 | 1.0000 | I |
| 15-B | 13.6 | 0.4 | 3.2 | 0.9982 | II |
| 15-C | 13.5 | 0.3 | 3.0 | 0.9987 | II |
| 11-A | 6.8 | 0.4 | 3.8 | 0.9927 | II |
| 15-D | 11.5 | 0.5 | 4.7 | 0.9957 | III |
| 15-E | 10.4 | 0.4 | 4.4 | 0.9968 | III |
| 11-A | 6.9 | 0.2 | 5.0 | 0.9981 | III |
| 15-C | 10.4 | 0.5 | 4.7 | 0.9960 | III |
| 11-A | 9.4 | 0.3 | 3.8 | 0.9993 | IV |
| 15-C | 12.6 | 0.2 | 3.8 | 0.9997 | IV |
| 15-F | 11.0 | 0.4 | 3.3 | 0.9988 | IV |
| 15-G | 10.1 | 0.7 | 4.3 | 0.9946 | IV |
| 15-H | 7.5 | 0.7 | 4.8 | 0.9914 | IV |
| 15-I | 6.1 | 0.5 | 4.5 | 0.9931 | IV |
| 15-J | 7.3 | 0.4 | 1.8 | 0.9840 | V |
| 15-K | 5.3 | 0.2 | 6.7 | 0.9915 | V |
| 11-A | 6.2 | 0.3 | 2.9 | 0.9885 | V |

Note to Table 14B:
R² = correlation coefficient for linear least square fit.

The data show that the skin permeation rate of the drug was sustained in a range of about 6 to 13.5 μg/cm²/hr. A sustained skin permeation rate in the range of about 6 to about 12 μg/cm²/hr. was judged useful for a 4-day patch regimen, and was provided by all of the patch formulations.

Patch Ex. 15-J was generally preferred. In stability tests, patch Ex. 15-J, stored in unpackaged form in a chamber at an ambient temperature of about 40° C. and relative humidity of about 75% (referred to as the 40/75 storage conditions) remained free of drug seed crystals for more than 21 days. Under the same 40/75 storage conditions, patch Ex. 11-A showed evidence of drug seed crystals within 24 hours, and patch Exs. 15 (A-E) remained crystal free for up to 14 days storage.

### EXAMPLE 16

This example illustrates drug-in-adhesive embodiments containing granisetron base, combinations of the penetration enhancers, laureth-4, mineral oil, PVP, and various adhesive transdermal patches prepared therefrom. Transdermal patches were prepared by general Method IV using a silicone-coated PET release liner (MEDIRELEASE® 2249) and occlusive backing film (SCOTCHPAK™ 9732). The % by weight of drug and penetration enhancer in the substantially dried DIA matrix is shown in Table 15.

TABLE 15
Weight Percent

| | | Laureth-4 | Mineral Oil | PVP | |
|---|---|---|---|---|---|
| | Granisetron | (N.2, | (N.4, | (N.2, | Adhesive* |
| Ex. | Base | Table 14) | Table 14) | Table 12) | q.s. to 100% |
| 16-A | 4 | 2 | 5 | 5 | DT-2979 |
| 16-B | 4 | 2 | 5 | 5 | DT-2196 |
| 16-C | 4 | 2 | 5 | 5 | DT-2100 |
| 16-D | 4 | 2 | 5 | 5 | DT-9301 |
| 16-E | 4 | 2 | 5 | 5 | GMS-3071 |
| 16-F | 4 | 2 | 5 | 5 | GMS-3067 |
| 16-G | 4 | 2 | 5 | 5 | GMS-9073 |
| 16-H | 4 | 2 | 5 | 5 | GMS-9083 |
| 16-I | 4 | 2 | 5 | 5 | GMS-737 |
| 16-J | 4 | 2 | 5 | 5 | GMS-788 |

Notes to Table 15:
*DT = DURO-TAK ®
GMS = GELVA ® GMS.

### EXAMPLE 17

This example illustrates drug-in-adhesive compositions containing varying amounts and combinations of the penetration enhancers, laureth-4, mineral oil, PVP, and transdermal patches prepared therefrom. Transdermal patches were prepared by general Method IV using a silicone-coated PET release liner (MEDIRELEASE® 2249) and occlusive backing film (SCOTCHPAK™ 9732). The % by weight of drug and penetration enhancer in the substantially dried DIA matrix is shown in Table 16 compared to patch Ex. 15-J.

TABLE 16
Weight Percent

| | | | | | Adhesive |
|---|---|---|---|---|---|
| Patch | Granisetron | Laureth-4 | Mineral Oil | PVP | DURO-TAK ® |
| Ex. | Base | (N. 2, Table 14) | (N. 2, Table 14) | (N. 2, Table 12) | 87-2516 |
| 17-A | 4 | None | 5 | 5 | q.s. to 100% |
| 17-B | 4 | None | None | None | q.s. to 100% |
| 17-C | 4 | 2 | None | 5 | q.s. to 100% |
| 17-D | 4 | 2 | 5 | None | q.s. to 100% |
| 17-E | 4 | None | 5 | 2.5 | q.s. to 100% |
| 15-J | 4 | 2 | 5 | 5 | q.s. to 100# |

The in vitro skin permeation rates were obtained by Method II, HPLC Method C (Table A) for transdermal patches prepared with compositions of Exs. 17-(B-E) were compared against the skin permeation rate of a transdermal patch of Ex. 15-J. FIG. 4 is a graphical representation of the average μg/cm²/hr. in vitro skin permeation over a time period of about 96 hours. The skin permeation rate in micrograms/cm²/hour is shown in Table 16A along with the lag time (hours).

TABLE 16A

| Patch | Skin Permeation | Standard | Lag Time | |
|---|---|---|---|---|
| Ex. | Rate (μg/cm²/hr.) | Error | (hours) | R² |
| 15-J | 11.10 | 0.93 | −4.16 | 0.97 |
| 17-E | 6.62 | 0.28 | −0.74 | 0.99 |
| 17-B | 6.17 | 0.30 | −1.32 | 0.99 |
| 17-C | 10.53 | 0.92 | −4.20 | 9.96 |
| 17-D | 10.19 | 0.86 | −3.81 | 9.97 |

Notes to Table 16A:
(a) Relative to Patch Ex. 12-A.
R² = correlation coefficient for linear least square fit.

The in vitro skin permeation rate data show that the transdermal patch Exs. 17-C, 17-D and 15-J containing penetration enhancers uniformly control a substantially increased drug release over that of the transdermal patch Ex. 17-B containing no penetration enhancer. The in vitro skin permeation rate of granisetron from the transdermal patch Ex. 15-J with time sustained more than 8 micrograms/cm²/hours for a period of up to about 72 hours and then gradually reduced to a rate of about 4 micrograms/cm² at about 96 hours.

Drug release rates obtained by Method I, HPLC Method C (Table A) for transdermal patches prepared with composition Exs. 17 (A-D) were compared to the drug release rate from a transdermal patch of Ex. 15-J (which contains 2% by weight laureth-4 and is otherwise compositionally similar to transdermal Patch Ex. 17-A). The results from duplicate samples over a 24-hour time period are shown in Table 16B, with the average drug release graphically represented in FIG. 5 as a plot of micrograms per square centimeter versus time (Square-Root-of-Time (hours^{1/2})).

TABLE 16B

| Patch | Drug Release Rate | Standard | Lag Time | |
|---|---|---|---|---|
| Ex. | (μg/cm²/hr.^{1/2}) | Error | (hours^{1/2}) | R² |
| 15-J | 143.0521 | 0.6111 | 0.0063 | 0.9999 |
| 17-A | 106.2236 | 1.0471 | −0.0169 | 0.9996 |
| 17-B | 81.4618 | 0.7981 | 0.0206 | 0.9996 |
| 17-C | 114.3484 | 1.7685 | 0.0409 | 0.9990 |
| 17-D | 126.3079 | 1.6018 | 0.0482 | 0.9994 |

The results indicated that the penetration enhancers promoted drug release. The highest drug release rate was achieved with transdermal patch Ex. 15-J and the lowest release rate with the transdermal patch Ex. 17-B (which contained no penetration enhancers).

The tack adhesion of six samples of transdermal patch Ex. 15-J and four samples each of transdermal patch Exs. 17 (A-D) was determined by Method III and the results are shown in Table 16C.

TABLE 16C
Tension Peak (N/0.2 cm²)

| Test | Patch Ex. | Patch Ex. | Patch Ex. | Patch Ex. | Patch Ex. |
|---|---|---|---|---|---|
| Sample # | 15-J | 17-A | 17-B | 17-C | 17-D |
| 1 | 4.77 | 5.88 | 7.65 | 7.28 | 5.47 |
| 2 | 5.76 | 5.69 | 7.93 | 6.87 | 4.65 |
| 3 | 4.67 | 5.56 | 7.86 | 5.08 | 4.82 |
| 4 | 4.98 | 6.24 | 7.98 | 6.99 | 4.96 |
| 5 | 4.95 | — | — | — | — |
| 6 | 5.26 | — | — | — | — |
| Average | 5.065 | 5.84 | 7.86 | 6.56 | 4.98 |
| Std Dev. | 0.396119 | 0.30 | 0.15 | 1.00 | 0.35 |

The average tack values show that transdermal patch Ex. 17-B had the highest tack adhesion, the patches with penetration enhancers had a peak tack value greater than 3N/0.2 cm² and were judged acceptable.

Two transdermal patches from each Example were stored, unpackaged, at ambient room temperature of about 25° C. and relative humidity of about 60% and separately at an elevated temperature of about 45° C. room storage and 75% relative humidity (accelerated aging) for a period of 10 days. The DIA matrix of the patches was then observed under a microscope for drug crystals. Under room storage conditions, none of the patches had drug crystals. Under accelerated aging conditions, however, drug crystals were observed in transdermal patch Exs. 17-A, 17-B and 17-D, with those in transdermal patch Ex. 17-B, which contains no penetration enhancer, being larger in size than those observed in transdermal patch Exs. 17-A and 17-D. Overall, the formulation of transdermal patch Ex. 15-J utilizing a combination of three penetration enhancers was preferred.

### EXAMPLE 18

This example illustrates the stability of transdermal patch embodiments of this invention under long-term ambient room storage conditions (about 25° C. and about 60% relative humidity (RH)) and accelerated aging storage conditions (about 45° C. and about 75% RH) over a storage period of about three months. These storage conditions generally conform to those acceptable to the International Conference on Harmonisation.

A 400 g batch of the composition of Example 15-J was prepared and transdermal patch embodiments were prepared having an active area of about 12.5 cm², using the silicone coated PET release liner (MEDIRELEASE® 2249) and the occlusive backing film (SCOTCHPAK™ 9732). The patches were individually packaged in aluminum foil pouches.

The stability of the transdermal patches was evaluated based on visual appearance, tack adhesion by Method III, and weight % of labeled drug content assayed after a storage period of two weeks, one month and three months. The results are summarized in Table 17.

TABLE 17
Patch Example 15-J Storage Conditions (° C./% RH)

| Storage Time: | | 2 Weeks | 1 Month | | 3 Months | |
|---|---|---|---|---|---|---|
| Test | Initial | 40° C./75 RH | 25° C./60 RH | 40° C./75 RH | 25° C./60 RH | 40° C./75 RH |
| Appearance | Conform | Conform | Conform | Conform | Conform | Conform |
| Tack Adhesion, | 5.07 | 6.03 | 5.505 | 5.403 | 5.3434 | 5.8266 |
| (N/0.2 cm²) | (±0.400) | (±0.590) | (±0.706) | (±1.405) | (±0.587) | (±0.500) |
| Drug Content | 104.3 | 101.9 | 106.7 | 105.5 | 103.52 | 102.4 |
| (Wt % Label) | (±4.2) | (±2.8) | (±3.1) | (±3.1) | (±3.9) | (±4.4) |

The data show that the tack adhesion, and % drug content of all the stored patches remained within the specified limits. All of the stored patches retained their rectangular shape and dimensions. No drug crystals were observed in the adhesive matrix with no sign of cold flow or residuals on the surface of the patch. No leakage was noted from the packaging.

The present invention has been described generally and with respect to preferred embodiments. It will be understood that modifications and variation of the disclosed composition and processes may be made without departing from the spirit and scope of the novel concept of the present invention.

1. A transdermal skin adhesive antiemesis composition having a drug-in-adhesive matrix and comprising, on a total composition weight basis,
an effective antiemesis amount of an antiemetic that is a selective 5-HT₃ receptor antagonist;
an amount of a skin permeation enhancer effective to provide a substantially uniform, sustained release of drug from the composition; and
an effective skin adhesive amount of a physiologically tolerable, water-insoluble pressure sensitive adhesive;
the antiemetic and permeation enhancer being incorporated in said adhesive to provide a drug-in-adhesive matrix.

2. The composition of claim 1 wherein the antiemetic is granisetron base or a pharmaceutically acceptable salt thereof.

3. The composition of claim 1 wherein the antiemetic is granisetron base and said permeation enhancer is capable of incorporating granisetron base therein in an amount in the range of at least 10 to at least about 200 micrograms per milliliter at an ambient room temperature in the range of about 20 to about 25° C.

4. The composition of claim 1, wherein at least one penetration enhancer is selected from the group consisting of a dipolar aprotic solvent, an aliphatic ester, a carboxylic ester that is a sorbitan derivative, a glycol, a polyol, an N,N-di(C₁-C₈) alkylamino substituted C₄-C₁₈) alkyl (C₂-C₁₈) carboxylic ester, an oil, a surfactant, and a polymer of 1-vinyl-2-pyrrolidone.

5. The composition claim 1, wherein the penetration enhancer comprises a homopolymer or vinyl acetate copolymer of 1-vinyl-2-pyrrolidone.

6. The composition of claim 5 wherein the homopolymer is polyvinylpyrrolidone.

7. The composition of claim 1, wherein the penetration enhancer comprises a surfactant.

8. The composition of claim 7 wherein the surfactant is laureth-4.

9. The composition of claim 1, wherein the penetration enhancer comprises a dipolar aprotic solvent.

10. The composition of claim 9, wherein the dipolar aprotic solvent is N-methyl-2-pyrrolidone.

11. The composition of claim 1, comprising at least two permeation enhancers, wherein at least one penetration enhancer is a dipolar aprotic solvent and another is a carboxylic ester that is a sorbitan derivative.

12. The composition of claim 11 wherein the dipolar aprotic solvent is N-methyl-2-pyrrolidone and the carboxylic ester is sorbitan monolaurate.

13. The composition of claim 1, comprising at least two permeation enhancers, wherein at least one penetration enhancer is polyvinylpyrrolidone and another is a polyol that is a polyethylene glycol.

14. The composition of claim 13 wherein the polyol is polyethylene glycol 400.

15. The composition of claim 1, comprising at least two permeation enhancers, wherein at least one penetration enhancer is polyvinylpyrrolidone and another is a glycol.

16. The composition of claim 1, comprising at least two penetration enhancers wherein at least one penetration enhancer is polyvinylpyrrolidone and another is a nonionic surfactant.

17. The composition of claim 16 wherein the surfactant is a polyoxyethylene fatty ether.

18. The composition of claim 17 wherein the surfactant is laureth-4.

19. The composition of claim 18 wherein the polyvinylpyrrolidone has a K-value in the range of about 29 to about 32.

20. The composition of claim 1, comprising at least two permeation enhancers, wherein at least one penetration enhancer is a mineral oil and another is a nonionic surfactant.

21. The composition of claim 1, comprising at least two permeation enhancers, wherein at least one penetration enhancer is a glycol and another is a nonionic surfactant.

22. The composition of claim 1, comprising granisetron base, polyvinylpyrrolidone, nonionic surfactant and mineral oil.

23. The composition of claim 22 wherein the nonionic surfactant is laureth-4.

24. The composition of claim 1, wherein the adhesive is selected from the group consisting of an acrylic adhesive, a rubber adhesive, a silicone adhesive, or a combination thereof.

25. The composition of claim 1, wherein the adhesive is substantially free from crystalline antiemetic.

26. A transdermal antiemesis system comprising an inert backing, and an effective antiemesis amount of the skin adhesive composition of claim 1 on one surface thereof, the skin adhesive composition providing a skin contacting surface.

27. The transdermal antiemesis system of claim 26, wherein the backing is occlusive and has a moisture vapor transmission rate of not more than about 200 g/m²/24 hours.

28. The transdermal antiemesis system of claim 26, wherein the backing comprises a polyester and ethylene vinyl acetate laminate.

29. The transdermal antiemesis system of claim 26, wherein the antiemetic is granisetron base, the permeation enhancer comprises N-methyl-2-pyrrolidone and sorbitan monolaurate, and the adhesive is an acrylic adhesive.

30. The transdermal antiemesis system of claim 26, wherein the antiemetic is granisetron base, the permeation enhancer comprises polyvinylpyrrolidone and laureth-4, and the adhesive is an acrylic adhesive.

31. The transdermal antiemesis system of claim 26, wherein the antiemetic is granisetron base, the permeation enhancer comprises polyvinylpyrrolidone, laureth-4, and mineral oil, and the adhesive is an acrylic adhesive.

32. The transdermal antiemesis system of claim 26 including a release liner on the skin contacting surface.

33. The transdermal antiemesis system of claim 32, wherein the release liner is a silicone coated polyester.

34. A method of delivering a therapeutic dose amount of antiemetic to a mammal comprising contacting the surface skin with the skin adhesive composition of a transdermal antiemesis system of claim 26, and maintaining the contact for a period sufficient to maintain effective therapy.

35. The method of claim 34, wherein the contact is effected at least once daily.

36. The method of claim 34, wherein the antiemetic is granisetron base and therapeutic dose is in the range of about 1 to about 2 mg granisetron released daily over a skin-contact period of up to about a week.

37. The transdermal antiemesis system of claim 26 in packaged form.

38. An article of manufacture comprising a kit for antiemesis therapy containing as the antiemesis composition the packaged composition of claim 37.

39. The article of manufacture of claim 38 further including instructional indicia for use therein.

40. The transdermal antiemesis system of claim 32 in packaged form.
